(19) United States
(12) Patent Application Publication
(10) Pub. No.: US 2018/0050107 A1
(43) Pub. Date: Feb. 22, 2018

(54) NEUROSTEROID COMPOSITIONS AND METHODS OF USE THEREOF

(71) Applicant: Janssen Pharmaceutica NV, Beerse (BE)

(72) Inventors: Thomas M DiMauro, Southboro, MA (US); Kevin Wildenhaus, Plymouth, MI (US); Michael J Fevola, Belle Mead, NJ (US); Tobias Johannes Fuetterer, Princeton, NJ (US)

(73) Assignee: Janssen Pharmaceutica NV, Beerse (BE)

(21) Appl. No.: 15/677,623

(22) Filed: Aug. 15, 2017

Related U.S. Application Data
(63) Continuation-in-part of application No. 15/354,114, filed on Nov. 17, 2016.
(60) Provisional application No. 62/402,439, filed on Sep. 30, 2016.
(60) Provisional application No. 62/375,676, filed on Aug. 16, 2016.

(51) Int. Cl. A61K 47/26; A61K 31/57; A61K 9/107

(52) U.S. Cl. CPC A61K 47/26; A61K 9/107; A61K 31/57

(57) ABSTRACT

Provided herein are compositions comprising neurosteroids and saponins, methods of making said compositions, and methods of utilizing said compositions to treat or prevent perinatal depression (PND) in a subject in need thereof.

### CROSS REFERENCE TO RELATED APPLICATIONS

This application is a continuation-in-part of and claims priority to U.S. patent application Ser. No. 15/354,114, filed Nov. 17, 2016, which claims priority to U.S. Provisional Patent Application No. 62/402,439, filed Sep. 30, 2016 and U.S. Provisional Patent Application No. 62/375,676, filed on Aug. 16, 2016. Each disclosure is incorporated herein by reference in its entirety.

### FIELD OF THE INVENTION

This invention relates to compositions comprising neurosteroids and saponins, methods of making said compositions, and methods of utilizing said compositions to treat or prevent perinatal (PND) depression in a subject in need thereof.

### BACKGROUND OF THE INVENTION

The loving connection between a mother and her baby is a special bonding that can benefit the baby not only in the present, but also well into the future. Bonding brings the mother and child closer together, and this positive attachment can enhance the baby's wellbeing and later development. Because a healthy bond between the mother and her newborn infant is crucial to the proper development of the child, loving efforts to strengthen that bond are highly valued. Some of the ways in which a healthy mother can show love for her child and promote this bonding is by experiencing joy at her child's smile and by providing appropriate attention to her child's needs.

It has been estimated that over 700,000 mothers are afflicted with postpartum depression (PPD) each year in the United States. PPD is considered to be a major depression, and is characterized by standard depressive features. Typical PPD symptoms include non-responsiveness towards the infant's needs and an absence of joy that is normally associated with healthy parent-child interaction and attachment. Because the first months of life are a critical period for an infant's proper cognitive and emotional development, the lack of attachment and attention towards the infant shown by the PPD mother may cause undesired effects in the child's future behaviors.

During pregnancy, the hormonal balance in the healthy expectant mother is such that she experiences extremely high levels of estrogen throughout her body. These levels of estrogen in the expectant mother may be up to 100 times the normal level. After the birth of the child, the estrogen level in the new mother rapidly decreases over the course of a few days and returns to the normal level of estrogen. Estrogen has been found to be critical to many normal neuronal processes, and has been positively associated with serotonin levels in the brain and brain plasticity. Therefore, and without wishing to be tied to a theory, it is believed that PPD may be caused by an extra-sensitive response in a subset of new mothers to the rapid withdrawal of estrogen from the mother's system.

Antidepressants are often one of the first lines of therapy against PPD. Conventional antidepressants such as tricyclics and selective serotonin reuptake inhibitors (SSRIs) are commonly prescribed for PPD. However, there are many problems associated with the use of these conventional antidepressants for PPD. First, these conventional antidepressants typically alleviate the PPD condition in no more than about 80% of the patients taking them. Second, even when successful, these conventional antidepressants typically take up to 8 weeks be effective. Third, the PPD mother can expect to experience the typical side effects associated with tricyclics and SSRIs. Side effects associated with tricyclics use include dry mouth, dry nose, blurred vision, decreased gastro-intestinal motility and secretion, leading to constipation, urinary retention, cognitive and/or memory impairment, and increased body temperature. Side effects associated with SSRI use include insomnia, weight gain and sexual dysfunction.

In addition, it has been found that virtually all of these conventional antidepressants are found in the mother's milk, and may be transferred to the infant during nursing. There has been little data on the effect of the nursing mother's antidepressant use upon the child's mental development. Rather than demonstrating safety, the literature appears to conclude that the risk to the nursing child posed by the mother's antidepressant use is outweighed by the risks associated with untreated PPD. However, in some cases, the transfer of some particular antidepressants to mother's milk has been so significant that some investigators have concluded that those particular antidepressants should be avoided by nursing mothers.

Sertraline (Zoloft) and paroxetine (Paxil) are the first-line antidepressants for treating PPD (Berle, Curr. Womens Health Rev. 2011 February; 7(1):28-34). No long term studies on the effects of these antidepressants on infants who receive their mother's milk have been conducted.

It has recently been reported by Sage Pharmaceuticals that their compound, SAGE-547, showed efficacy in a small double blind human trial. However, the SAGE-547 must be administered by an intravenous method, and so poses a problem with day-to-day compliance.

Therefore, one goal is to provide a GABA(A) delta agonist that can be administered non-invasively.

### BRIEF SUMMARY OF THE INVENTION

It has now been appreciated that there exists in nature several GABA(A) agonists that possess a self-assembling quality. This quality allows the skilled artisan to make self-assembled structures including gels, micelles, lamellar vesicles, multi-lamellar vesicles (MLVs), and liposomes from the natural GABA(A) delta agonists. Because MLVs, micelles and liposomes can be administered non-invasively through the oral, intranasal or pulmonary routes, the present compositions present an advantage over the prior art intravenous compositions.

In some embodiments, the natural GABA(A) delta agonist is presented in the form of micelles. Micelles provide an advantage in that they can be orally administered and that their small size evades detection by macrophages, which provides for an extended circulation time in the human vasculature.

In some embodiments, the natural GABA(A) delta agonist is presented in the form of liposomes. It is believed that the liposomal form provides an advantage during pulmonary administration of the GABA(A) delta composition. Liposomes are generally on the order of 100-200 nanometers (and so are categorized as fine particles), while micelles are much smaller at about 10-20 nm (and so are categorized as ultrafine particles). Because a substantially larger fraction of micelles are exhaled after pulmonary administration, liposomes provide an advantage (over micelles) in that their relatively larger size provides a much more efficient pulmonary administration. Liposomes can also deliver hydrophilic molecules housed in their aqueous cores.

In some embodiments, the natural GABA(A) delta agonist is presented in the form of multi-lamellar vesicles (MLVs). It is believed that the MLV form provides an advantage during pulmonary administration of the GABA(A) delta composition. MLVs can be made to a size on the order of a few microns. Because a substantially larger fraction of micelles and liposomes are exhaled after pulmonary administration, MLVs provide an advantage (over micelles and liposomes) in that their relatively larger size provides a much more efficient pulmonary administration.

Therefore, in some embodiments, there is provided a composition comprising a plurality of mixed self-assemblies comprising i) at least 50 wt % of a cyclic molecule, and ii) at least 5 wt % of a natural GABA(A) delta agonist intercalated therein.

In other embodiments, there is provided a composition comprising a plurality of mixed self-assemblies comprising i) at least 50 wt % of an uncharged molecule, and ii) at least 5 wt % of a natural GABA(A) delta agonist intercalated therein.

In other embodiments, there is provided a composition comprising a plurality of mixed self-assemblies comprising i) at least 50 wt % of a glycosylated molecule, and ii) at least 5 wt % of a natural GABA(A) delta agonist intercalated therein. Preferably, each has at least a trihexacyclic structure.

In other embodiments, there is provided a composition comprising a plurality of mixed self-assemblies comprising i) at least 50 wt % of a phenolic molecule, and ii) at least 5 wt % of a natural GABA(A) delta agonist intercalated therein. Preferably, each has a biphenyl structure, more preferably a biphenolic structure.

In some embodiments, there is provided a composition comprising (a) a neurosteroid; and (b) a saponin in an amount effective to form a self-assembled structure incorporating the neurosteroid. The self-assembled structure can, for example be selected from the group consisting of a micelle, a gel, a liposome, a lamellar phase vesicle, and a multi-lamellar vesicle.

In certain embodiments, the saponin is selected from the group consisting of a soyasaponin, a quillaja saponin, and a ginsenoside saponin. The saponin can, for example, be at least about 0.1 wt %, at least about 0.5 wt %, or at least about 1 wt % relative to the total weight of the composition.

In certain embodiments, the neurosteroid is selected from the group consisting of an allopregnanolone, a tetrahydrodeoxycorticosterone (THDOC), and a progesterone. The composition comprises at least about 100 parts per million, at least about 200 parts per million, or at least about 300 parts per million of the neurosteroid.

In certain embodiments, the composition further comprises a pharmaceutically acceptable carrier or an adjuvant.

In certain embodiments, the composition has a permeation coefficient P of about 0.01 per hour to about 0.05 per hour, preferably about 0.01 per hour.

In other embodiments, there is provided methods of treating a perinatal depression (PND) in a subject in need thereof. The methods comprise administering to the subject a therapeutically effective amount of compositions according to embodiments of the invention. In certain embodiments, the therapeutically effective amount of the composition is administered topically, intravenously, orally, mucosally, or by inhalation. In certain embodiments, the therapeutically effective amount of the composition is administered in a single dose, once every 4 hours, preferably once every 8 hours, more preferably once every 12 hours, most preferably once every 24 hours or a longer period of time.

In certain embodiments, the therapeutically effective amount of the composition has a permeation coefficient P of about 0.01 per hour to about 0.05 per hour, preferably about 0.01 per hour. In certain embodiments, the therapeutically effective amount of the composition has a permeation coefficient P at least two times, at least five times, or at least ten times lower than a permeation coefficient P of a composition comprising a monomolecular neurosteroid or a cyclodextrin neurosteroid complex.

In other embodiments, there is provided methods of producing compositions according to embodiments of the invention. The methods comprise adding a solution of the neurosteroid dissolved in an organic solvent to a saponin solution under flow, wherein the neurosteroid is incorporated into the self-assembled structure. In certain embodiments, the organic solvent dissolves at least 0.1 wt %, at least 0.5 wt %, or at least 1 wt % of the neurosteroid. In certain embodiments, at least 1 wt %, at least 2.5 wt %, or at least 3 wt % of the organic solvent is soluble in water. In certain embodiments, the organic solvent is selected from the group consisting of ethanol, methanol, propanol, butanol, glycol, ethylene glycol, propylene glycol, butylene glycol, diethyl ether, and mixtures thereof.

### BRIEF DESCRIPTION OF THE DRAWINGS

The foregoing summary, as well as the following detailed description of preferred embodiments of the present application, will be better understood when read in conjunction with the appended drawings. It should be understood, however, that the application is not limited to the precise embodiments shown in the drawings.

FIG. 1 shows a comparative picture of a sample without allopregnanolone crystals present (left) and a sample with crystals present (right). The left vial contains 4.6 wt % VaxSap saponin in deionized (DI) water, and the right vial contains 4.6% VaxSap saponin in DI water with 1600 ppm allopregnanolone crystals.

FIG. 2 shows a standard curve of allopregnanolone concentrations for Arbor Assays™ DetectX® Allopregnanolone Enzyme Immunoassay.

FIG. 3 shows a graph of the relative amounts of allopregnanolone released over time for systems with (square symbols) and without (round symbols) saponin. The lines are fits to the data using formula 1.

### DETAILED DESCRIPTION OF THE INVENTION

Various publications, articles and patents are cited or described in the background and throughout the specification; each of these references is herein incorporated by reference in its entirety. Discussion of documents, acts, materials, devices, articles or the like which has been included in the present specification is for the purpose of providing context for the invention. Such discussion is not an admission that any or all of these matters form part of the prior art with respect to any inventions disclosed or claimed.

Unless defined otherwise, all technical and scientific terms used herein have the same meaning as commonly understood to one of ordinary skill in the art to which this invention pertains. Otherwise, certain terms used herein have the meanings as set forth in the specification.

It must be noted that as used herein and in the appended claims, the singular forms “a,” “an,” and “the” include plural reference unless the context clearly dictates otherwise.

Unless otherwise stated, any numerical values, such as a concentration or a concentration range described herein, are to be understood as being modified in all instances by the term “about.” Thus, a numerical value typically includes ±10% of the recited value. For example, a concentration of 1 mg/mL includes 0.9 mg/mL to 1.1 mg/mL. Likewise, a concentration range of 1% to 10% (w/v) includes 0.9% (w/v) to 11% (w/v). As used herein, the use of a numerical range expressly includes all possible subranges, all individual numerical values within that range, including integers within such ranges and fractions of the values unless the context clearly indicates otherwise.

Unless otherwise indicated, the term “at least” preceding a series of elements is to be understood to refer to every element in the series. Those skilled in the art will recognize, or be able to ascertain using no more than routine experimentation, many equivalents to the specific embodiments of the invention described herein. Such equivalents are intended to be encompassed by the invention.

As used herein, the terms “comprises,” “comprising,” “includes,” “including,” “has,” “having,” “contains” or “containing,” or any other variation thereof, will be understood to imply the inclusion of a stated integer or group of integers but not the exclusion of any other integer or group of integers and are intended to be non-exclusive or open-ended. For example, a composition, a mixture, a process, a method, an article, or an apparatus that comprises a list of elements is not necessarily limited to only those elements but can include other elements not expressly listed or inherent to such composition, mixture, process, method, article, or apparatus. Further, unless expressly stated to the contrary, “or” refers to an inclusive or and not to an exclusive or. For example, a condition A or B is satisfied by any one of the following: A is true (or present) and B is false (or not present), A is false (or not present) and B is true (or present), and both A and B are true (or present).

As used herein, the conjunctive term “and/or” between multiple recited elements is understood as encompassing both individual and combined options. For instance, where two elements are conjoined by “and/or”, a first option refers to the applicability of the first element without the second. A second option refers to the applicability of the second element without the first. A third option refers to the applicability of the first and second elements together. Any one of these options is understood to fall within the meaning, and therefore satisfy the requirement of the term “and/or” as used herein. Concurrent applicability of more than one of the options is also understood to fall within the meaning, and therefore satisfy the requirement of the term “and/or.”

As used herein, the term “consists of,” or variations such as “consist of” or “consisting of,” as used throughout the specification and claims, indicate the inclusion of any recited integer or group of integers, but that no additional integer or group of integers can be added to the specified method, structure, or composition.

As used herein, the term “consists essentially of,” or variations such as “consist essentially of” or “consisting essentially of,” as used throughout the specification and claims, indicate the inclusion of any recited integer or group of integers, and the optional inclusion of any recited integer or group of integers that do not materially change the basic or novel properties of the specified method, structure or composition. See M.P.E.P. §2111.03.

The words “right”, “left”, “lower” and “upper” designate directions in the drawings to which reference is made.

It should also be understood that the terms “about,” “approximately,” “generally,” “substantially” and like terms, used herein when referring to a dimension or characteristic of a component of the preferred invention, indicate that the described dimension/characteristic is not a strict boundary or parameter and does not exclude minor variations therefrom that are functionally the same or similar, as would be understood by one having ordinary skill in the art. At a minimum, such references that include a numerical parameter would include variations that, using mathematical and industrial principles accepted in the art (e.g., rounding, measurement or other systematic errors, manufacturing tolerances, etc.), would not vary the least significant digit.

As used herein, “subject” means any animal, preferably a mammal, most preferably a human. The term “mammal” as used herein, encompasses any mammal. Examples of mammals include, but are not limited to, cows, horses, sheep, pigs, cats, dogs, mice, rats, rabbits, guinea pigs, monkeys, humans, etc., more preferably a human.

As used herein, the term “therapeutically effective amount” in the context of administering a therapy to a subject refers to the amount of the composition which has a prophylactic and/or therapeutic effect(s). In certain embodiments, a “therapeutically effective amount” in the context of administration of the composition to the subject refers to the amount of the composition, which is sufficient to achieve a reduction or amelioration in the severity of the perinatal depression in the subject, a reduction in the duration of the perinatal depression in the subject, and/or a prevention of the perinatal depression in the subject. In certain embodiments, the therapeutically effective amount of the composition does not completely treat the perinatal depression in the subject, but rather reduces or ameliorates the symptoms and severity of the perinatal depression in the subject as compared to an untreated subject.

As used herein, “neurosteroid” means any endogenous or exogenous steroid that rapidly alters neuronal excitability through interaction with ligand-gated ion channels and other cell surface receptors. A neurosteroid can, for example, be a steroid that is synthesized in the brain or is synthesized by an endocrine gland that then ultimately reaches the brain through the bloodstream and has an effect on the brain function. Examples of neurosteroids include, but are not limited to, pregnanes (e.g., dihydroxyprogesterone, allopregnanolone, pregnanolone, dihydrodeoxycorticosterone, and tetrahydrodeoxycorticosterone), androstanes (e.g., androstenol, androsterone, androstanediol, etiocholanolone), 3β-hydroxysteroids (e.g., pregnenolone sulfate, dehydroepiandrosterone, and dehydroepiandrosterone sulfate), and pheromones.

As used herein, “incorporating” as used with respect to “a self-assembled structure incorporating a neurosteroid,” means encapsulating, embedding, and any other way for a self-assembled structure to assemble with the neurosteroid.

GABA(A) Delta Agonists and Compositions Thereof

For the purposes of the present invention, a GABA(A) delta agonist increases a GABA(A) current at least 10% at 100 uMol/L.

In some embodiments, the GABA(A) delta agonist is derived from a plant. In others, the agonist is endogenous to mammals. In others, the agonist is endogenous to humans.

In some embodiments, the self-assembled structure is selected from the group consisting of a micelle, a gel, a liposome, a lamellar phase vesicle, a multi-lamellar vesicle (MLV), and a solid lipid nanoparticle.

In some embodiments, each of the cyclic structures and the agonist contains a biphenyl structure. These common aromatic structures allow the agonist to nest within opposed biphenyls of the cyclic superstructure (i.e., the biphenyl of the agonist intercalates between the biphenyls of the cyclic superstructure). This intercalation is carried out due to the pi-pi bonding between the aromatic components of the biphenyl structures.

In some embodiments, the cyclic molecule is ellagic acid, a urolithin, a punicallagin or mixtures thereof. In some embodiments, the cyclic molecule is ellagic acid, a urolithin or mixtures thereof and the agonist is a lignan selected from the group consisting of honokiol, magnolol, and mixtures thereof. Preferably, the cyclic molecule is ellagic acid and the agonist is a lignan selected from the group consisting of honokiol, magnolol, and mixtures thereof.

In some embodiments, the cyclic molecule is unsaturated. In some embodiments, each of the unsaturated cyclic molecule and the agonist has at least three cyclohexylic rings. These common rings allow the agonist to nest within opposed rings of the unsaturated cyclohexylic superstructure.

In some embodiments, the self-assembled structures comprise at least 15 wt % of the natural GABA(A) delta agonist. Preferably, the self-assembled structures comprise at least 30 wt % of the natural GABA(A) delta agonist.

In some embodiments, the self-assembled structures comprise at least 65 wt % of the cyclic molecule, preferably, at least 80 wt % of the cyclic molecule.

In some embodiments, the cyclic molecule is at least bicyclic (i.e., has at least two rings).

In some embodiments, the cyclic molecule is a saponin and the agonist is a neurosteroid. In some embodiments, provided is a composition comprising (a) a neurosteroid; and (b) a saponin in an amount effective to form a self-assembled structure incorporating the neurosteroid. In certain embodiments, the saponin is selected from the group consisting of a soyasaponin, quillaja saponin. and a ginsenoside, and the neurosteroid is selected from the group consisting of allopregnanolone, tetrahydrodeoxycorticosterone (THDOC), and progesterone.

In some embodiments, the self-assembled structure is selected from the group consisting of a micelle, a gel, a liposome, a lamellar phase vesicle, a multi-lamellar vesicle (MLV), and a solid lipid nanoparticle.

In some embodiments, the saponin is at least about 0.1 wt %, at least about 0.5 wt %, at least about 1 wt %, at least about 2 wt %, or at least about 5 wt % relative to the total weight of the composition. The saponin can, for example, be up to about 50 wt %, up to about 20 wt %, up to about 10 wt %, up to about 5 wt %, or up to about 1 wt % relative to the total weight of the composition. The saponin can, for example, be about 0.1 wt % to about 5 wt %, about 0.5 wt % to about 4 wt %, about 1 wt % to about 3 wt %, or any number in between relative to the total weight of the composition.

In some embodiments, the self-assembled structure contains at least 5 mol % neurosteroid, preferably at least 10 mol %, more preferably at least 20 mol %, more preferably at least 30 mol %, more preferably at least 40 mol %.

In some embodiments, the composition comprises at least about 100 parts per million, at least about 200 parts per million, at least about 300 parts per million, at least about 500 parts per million, at least about 1000 parts per million, or at least about 5000 parts per million of the neurosteroid. The neurosteroid can, for example, be about 100 parts per million to about 5000 parts per million, about 200 parts per million to about 1000 parts per million, about 300 parts per million to about 500 parts per million, or any number in between in the composition.

In some embodiments, the composition has a permeation coefficient P of about 0.001 per hour to about 0.5 per hour, about 0.005 per hour to about 0.1 per hour, about 0.01 per hour to about 0.05 per hour, or any number in between In some embodiments, the permeation coefficient P is about 0.001 per hour, about 0.005 per hour, about 0.01 per hour, about 0.02 per hour, about 0.03 per hour, about 0.04 per hour, about 0.05 per hour, about 0.1 per hour, or about 0.5 per hour. In some embodiments, the permeation coefficient P is at least two times, at least five times, at least ten times, or at least fifty times lower than a permeation coefficient P of a composition comprising a monomolecular neurosteroid or a cyclodextrin neurosteroid complex.

Allopregnanolone and THDOC are highly potent GABA(A) delta agonists. THDOC increases the GABA current by at least 700% at a concentration of 1 μMol/L. See, e.g., Wohlfarth, J. Neurosci., 2002, 22, 5, 1541-9. Allopregnanolone potentiates rat cerebellar GABA delta subunits in the nanomolar range. Fodor, Neurosci. Lett., 2005, 383, (1-2), 127-130.

Allopregnanolone is endogenous to human and rises in plasma concentration during pregnancy from less than 5 ng/ml to about 50 ng/ml. Luisi, J. Clin. Endocrinol. Metab., 2000, July 85, 7, 2429-33. Therefore, it can be administered safely to a mother without concern for the health of the breastfeeding infant.

THDOC is endogenous to human and exists in concentrations as high as 0.5 ng/ml in the plasma of humans. Brambilla, Psychiatry Research, 135, 2005, 185-190. Therefore, it can be administered safely to a mother without concern for the health of the breastfeeding infant.

Progesterone is endogenous to humans and rises in plasma concentration during pregnancy from less than 10 ng/ml to about 150 ng/ml. Luisi, J. Clin. Endocrinol. Metab., 2000, July 85, 7, 2429-33. Therefore, it can be administered safely to a mother without concern for the health of the breastfeeding infant.

In one embodiment, a self-assembled allopregnanolone/soyasaponin mixed micellar structure is made substantially in accordance with the recipe for making an ginsenoside micelles disclosed in Xiong, Intern. J. Pharmaceutics, 360 (2008) 191-196. In one such prophetic embodiment, a series of working solutions are prepared by dissolving a 10 mol % allopregnanolone/90 mol % soyasaponin mixture in water and physiologic saline to produce 0.1-0.6 mg/ml solutions. These working solutions are then filtered through a 0.8 um filter. Surface tension is then measured to identify the critical micellar concentration CMC. The micellar solutions are then subject to evaporation to obtain dry mixed micelles.

In some embodiments, provided herein are methods of producing compositions according to embodiments of the invention. The methods are based on convection-driven solvent-to-water complexation (CSWC) methods. The methods comprise adding a solution of the neurosteroid dissolved in an organic solvent to a saponin solution under flow, wherein the neurosteroid is incorporated into the saponin self-assembled structure.

The organic solvent can, for example, dissolve at least 0.01 wt %, at least 0.1 wt %, at least 0.5 wt %, at least 1 wt %, at least 5 wt %, or at least 10 wt % of the neurosteroid. The organic solvent can, for example, dissolve about 0.01 wt % to about 10 wt %, about 0.1 wt % to about 5 wt %, about 0.5 wt % to about 1 wt %, or any number in between, of the neurosteroid.

In some embodiments, at least 0.1 wt %, at least 0.5 wt %, at least 1 wt %, at least 2.5 wt %, at least 3 wt %, at least 5 wt %, or at least 10 wt % of the organic solvent is soluble in water. About 0.1 wt % to about 10 wt %, about 0.5 wt % to about 5 wt %, about 1 wt % to about 3 wt %, or any amount in between, of the organic solvent is soluble in water.

In some embodiments, the organic solvent is selected from the group consisting of ethanol, methanol, propanol, butanol, glycol, ethylene glycol, propylene glycol, butylene glycol, diethyl ether, and mixtures thereof.

In some embodiments, the allopregnanalone is disposed in an alcohol solution (such as 95% ethanol) prior to its mixing with the saponin, as doing so increases the solubility of the allopregnanalone in the solution and allows for its facile intercalation during the fabrication of the saponin self-assembly.

In one embodiment, a self-assembled allopregnanolone/ginsenoside mixed micellar structure is made substantially in accordance with the recipe for making an ginsenoside micelles disclosed in Xiong, Intern. J. Pharmaceutics, 360 (2008) 191-196. In one such prophetic embodiment, a series of working solutions are prepared by dissolving a 10 mol % allopregnanolone/90 mol % ginsenoside mixture in water and physiologic saline to produce 1-100 mg/ml solutions. These working solutions are then filtered through a 0.8 um filter. Surface tension is then measured to identify the critical micellar concentration CMC. The micellar solutions are then subject to evaporation to obtain dry mixed micelles.

In one embodiment, a self-assembled allopregnanolone/quillaja saponin mixed micellar structure is made substantially in accordance with the recipe for making an ginsenoside micelles disclosed in Xiong, Intern. J. Pharmaceutics, 360 (2008) 191-196. In one such prophetic embodiment, a series of working solutions are prepared by dissolving a 10 mol % allopregnanolone/90 mol % quillaja saponin mixture in water and physiologic saline to produce 1-100 mg/ml solutions. These working solutions are then filtered through a 0.8 um filter. Surface tension is then measured to identify the critical micellar concentration CMC. The micellar solutions are then subject to evaporation to obtain dry mixed micelles.

In the neurosteroid/saponin embodiments, a thin film hydration method can be used to make multi-lamellar vesicles (MLVs) and liposomes.

In order to make self-assembled MLVs, DBC/TTC is first dispersed in an organic solvent in a rotatory evaporator flask, and the solvent is evaporated to leave a thin film on the bottom of the flask. The film is then hydrated with water, and the flask is gently agitated to form the MLVs.

In another embodiment, a self-assembled MLV is made as above, and then shear is imparted upon the solution. In some embodiments, the shear is produced by a high speed blender. In other embodiments, the shear is produced by a commercially available ultrasonic cleaner.

The self-assembled allopregnanolone/ginsenoside mixed self-assembled structures are useful because ginsenosides are also known as useful for treating diabetes. Thus, with a single structure the clinician can treat both postpartum depression and gestational diabetes in the same mother.

It has been reported that self-assembled ginsenoside micelles can be tuned to have release rates from days to months. Xiong, Int. J. Pharm. 2008 Aug. 6; 360(1-2):191-6. The tuning is performed by varying the concentration of the ginsenoside in the initial solution, with higher concentrations leading to slower release rates. Without wishing to be tied to a theory, it is believed self-assembled vesicles consisting essentially of a glycosylated saponins (such as soyasaponin) can likewise be tuned to have release rates from days to months, with the tuning being performed by varying the concentration of the local anaesthetic in the initial solution, with higher concentrations leading to slower release rates.

Therefore, in some embodiments that provide for extended release rates, the self-assembled micelle of allopregnanolone/soyasaponin is made by dispersing allopregnanolone/soyasaponin in water at a concentration above 1 mg/ml, preferably above 5 mg/ml, preferably above 20 mg/ml, preferably above 30 mg/ml, preferably above 50 mg/ml, preferably above 70 mg/ml, preferably above 80 mg/ml, preferably above 90 mg/ml, preferably above 100 mg/ml.

Likewise, in some embodiments that provide for extended release rates, the self-assembled micelle of allopregnanolone/ginsenoside is made by dispersing allopregnanolone/ginsenoside in water at a concentration above 1 mg/ml, preferably above 5 mg/ml, preferably above 20 mg/ml, preferably above 30 mg/ml, preferably above 50 mg/ml, preferably above 70 mg/ml, preferably above 80 mg/ml, preferably above 90 mg/ml, preferably above 100 mg/ml.

Xiong teaches that loading determines release rate. In some embodiments, the loading of the self-assembly is targeted to provide a release rate that corresponds to about a 100% release in about 24 hours and a 50% rate at about 12 hours. This loading and corresponding rate would enable the mother to take only one dose a day (and thereby promote compliance more than a multiple-dose-per-day routine) while still enabling a habit-forming routine of taking one dose per day (thereby promote compliance more than a one-dose-every-few-days routine).

According to Alexeev, Neuropharmacology. 2012 June; 62(8):2507-14, honokiol and magnolol are very potent GABA(A) delta agonists, as each increases the GABA current by at least about 800% at a concentration of about 10 uMol/L.

Honokiol and magnolol are found in the fruit and bark of the magnolia tree. Honokiol has been suggested to treating post-natal pain in infants (Woodbury, J Nat Prod. 2015 Nov. 25; 78(11):2531-6). Therefore, it appears to be a good candidate for safe administration to a mother without much concern for the health of the breastfeeding infant.

In one embodiment, honokiol/ellagic acid self-assembled structures are made substantially in accordance with the recipe for making an ellagic acid self-assembled structure disclosed in Frayne, Materials Express, 2, 4, 2012 335-343. In one such prophetic embodiment, 10% honokiol/90% ellagic acid assemblies are prepared in aqueous solution at pH 7. A stock solution of 50 mL of 9 mM ellagic acid and 1 mM honokiol was dissolved in 0.1 M NaOH and filtered. To the filtrate, 0.1 M solution of citric acid was added to adjust the pH value of the solution to 8. In some embodiments for making elongated sandwich structures, the above mixture is allowed to grow for a maximum of 15 minutes (thereby preventing polymerization from occurring). The formed assemblies are sonicated for thirty minutes, washed and deionized with water, and centrifuged twice at 15000 rpm before further analysis.

The self-assembled honokiol/ellagic acid mixed micellar structures are useful because ellagic acid is also known as useful for treating diabetes. Thus, with a single structure the clinician can treat both postpartum depression and gestational diabetes in the same mother.

These self-assembled combinations can be administered through oral, intranasal, buccal or pulmonary routes. The pulmonary route is preferred, as above.

It is further recognized that there are many other additional combinations of natural molecules (both phytochemicals and metabolites) whose first molecule has sufficient surfactant-like quality to form the superstructure of self-assemblies such as micelles, MLVs, large unilamellar vesicles (LUVs), liposomes, cylinders, fibers and discs, and a second active molecule that has sufficient structure to nest neatly in the superstructure to provide enhanced bonding and therefore a slow release rate. A listing of some of these combinations of molecules is provided in Table 1. These self-assembled combinations can be administered through oral, intranasal, buccal or pulmonary routes. The pulmonary route is preferred, as above.

Each of myrtenol and verbenol is also a GABA(A) delta agonist at 100 uM. Van Brederode, Neuroscience Letters, 628, (2016) 91-97. Each of myrtenol and verbenol is a major metabolite of alpha-pinene (pine nuts). Schmidt, Arch. Toxicol., 2015, Dec. 17, and has been determined to be Generally Regarded as Safe (GRAS) by the FDA (Duke, 2000).

In one embodiment, a self-assembled verbenol or myrtenol micelle is made substantially in accordance with the recipe for making a camphor micelle disclosed (Turina, Biophysical Chemistry, 122, 2006, 101-113). In one such prophetic embodiment, verbenol is dispersed in water at a concentration above 0.01 mM.

In another embodiment, a self-assembled verbenol liposome is made by first providing verbenol dispersed in an organic solvent, rotary evaporating the solvent to form a thin film on the flask bottom, hydrating the film with agitation to form MLVs. Liposomes can be made by further imparting shear upon the solution. In some embodiments, the shear is produced by a high speed blender. In other embodiments, the shear is produced by a commercially available ultrasonic cleaner. LUVs can be made as above.

It is further recognized that there are many other additional natural molecules (both phytochemicals and metabolites) that, like the verbenol and myrtenol molecules, also have sufficient surfactant-like quality to form self-assembled structures such as micelles, MLVs, LUVs, liposomes, cylinders, fibers and discs all by themselves. That is, the self-assembly consists essentially of the natural molecule. A listing of some of these molecules is provided in Table 2. These self-assembled structures can be administered through oral, intranasal, buccal or pulmonary routes. The pulmonary route is preferred, as above. In some embodiments, the self-assembly consists essentially of a phytochemical. In some embodiments, the self-assembly consists essentially of an endogenous molecule. In some embodiments, the self-assembly consists essentially of a human metabolite.

The thin film hydration method can be used to make MLVs, large unilamellar vesicles (LUVs) and liposomes from the combinations and molecules listed in Tables 1 and 2.

In order to make self-assembled MLVs, the surfactant is first dispersed in an organic solvent in a rotatory evaporator flask, and the solvent is evaporated to leave a thin film on the bottom of the flask. The film is then hydrated with water, and the flask is gently agitated to form the MLVs.

In another embodiment for making LUVs, a self-assembled MLV is made as above, and the MLVs are then extruded through a properly sized filter to form the LUV.

In another embodiment, a self-assembled MLV is made as above, and then shear is imparted upon the solution to produce liposomes. In some embodiments, the shear is produced by a high speed blender. In other embodiments, the shear is produced by a commercially available ultrasonic cleaner.

In some embodiments, oxytocin or an analog thereof is provided in the water core of the liposome in an amount effective for treating postpartum depression (PPD).

TABLE 1

| | | | Rationale for self- |
|---|---|---|---|
| Molecule | Mode of Action | Application | assembling behavior |
| Magnolol | GABA(A) | Postpartum | Intercalation in ellagic acid, |
| | delta agonist | depression; | which forms a sandwich (1) |
| | | cancer (HIF) | |
| Honokiol | GABA(A) | Postpartum | Intercalation in ellagic acid, |
| | delta agonist | depression; | which forms a sandwich (1) |
| | | cancer | |

(1) Barnaby, Nanosci. Nanotech. 9: 7579-86 (2011)

TABLE 2

| | | | Rationale for self- |
|---|---|---|---|
| Molecule | Mode of Action | Application | assembling behavior |
| Borneol | GABA(A) delta agonist | Depression | Looks like camphor |
| Sulforaphane | Nrf2 | Autism; breast cancer | Similarity to octyl methyl |
| | | metastasis; COPD | sulfoxide (2) |
| Perillyl alcohol | Ras inhibitor | Glioblastoma | Similarity to menthol |
| | | multiforme | |
| Allicin | Antibiotic | Ear infection | Similarity to sodium |
| | | | ricinolate (3) |
| 2-arachidonoyl | Endogenous cannabinoid | Postpartum | Similarity to ceramide, which |
| glycerol (2-AG) | | depression | forms a liposome (4) |
| Anandamide | Endogenous cannabinoid | Postpartum | Similarity to ceramide |
| | | depression | |
| Oleamide | Endogenous cannabinoid | Postpartum sleep | Similarity to ceramide |
| 17-hydroxy | Precursor to | Postpartum | Similarity to DHA (5); |
| docosahexanoic | neuroprotectin | depression; diabetes | similarity to ricinoleic acid |
| acid | | | |
| Hesperidin | Upregulates BDNF | Postpartum | Glycosylated flavonoid |
| | | depression; diabetes | |
| 1,8 cineole | Brain wave | Mood elevation | (6) |
| Ganglioside GM3 | Self-assembling liposome | Postpartum | Similarity to lecithin |
| | housing oxytocin | depression | |
| Hyperoside | Antidepressant; Beta2- | Postpartum | Glycosylated flavonoid |
| | adrenergic blocker | depression; metastatic | |
| | (Beta-blocker) | breast cancer | |
| Quercetin-3- | Beta2-adrenergic blocker | Metastatic breast | Similarity to hyperoside |
| glucuronide (Q3G) | (Beta-blocker) | cancer | |
| Rutin | Precursor of hyperoside | Metastatic breast | Double glycosylated |
| | | cancer | flavonoid |
| Propranolol | Beta2-adrenergic blocker | Metastatic breast | Classic polar cationic head |
| | (Beta-blocker) | cancer; hypertension | and lipophilic tail |
| Crocetin/crocin | Antidepressant | Postpartum | Bolaamphiphile with similar |
| | | depression | diacid structure (7) |
| 10- | Promotes neurogenesis of | Antidepressant; | Hydroxyl & carboxyl ends |
| hydroxydecenoic | neural stem cells | cancer | |
| acid | | | |
| Bisabolol oxide A | | | Intercalation with |
| | | | soyasaponin/gensenoside |
| Sphinosine-1- | Chemotactic agent for | Intradiscal injection | Similarity to lecithin |
| phosphate (S1P) | stem cells | for DDD; fusion | |
| | | agent | |
| 2-hydroxyoleic | Anticancer agent | Glioma; leukemia; | Classic polar head- |
| acid | | breast cancer; and | hydrophobic tail surfactant |
| | | colon cancer | structure |
| Oleuropein | Anticancer | Breast cancer | Classic glycosidic head- |
| | | | hydrophobic tail surfactant |
| | | | structure |

(2) Ioyota et al., Colloid Interface Sci. 299(1): 428-34 (2006)
(3) Shinde et al., Phys. Chem. 96: 5160-5 (1992)
(4) Park et al., Biochem. Biophys. Res. Commun. 435(3): 361-6 (2013)
(5) Mooibroek et al., Int. J. Radiat. Biol. Relat. Stud. Phys. Chem. Med. 42(6): 601-9 (1982).
(6) Turina et al., Biophys. Chem. 122(2): 101-13 (2006).
(7) Zhang et al., J. Colloid Interface Sci. 261(2): 417-22 (2003).

Without wishing to be tied to a theory, it is believed that perinatal depression (PND) is not a single condition, but rather is a heterogeneous disorder consisting of at least six different phenotypes. Presented herein is a portfolio of novel products, each of which provides a tailored pharmaceutical treatment for at least one of the six PND phenotypes. Because there is sensitivity to the possibility of transferring these products to the infant through breastfeeding, the tailored solutions use only molecules having extremely high safety profiles (i.e., nutriceuticals, their metabolites or endogenous molecules).

Table 3 provides a description of at least some of the hypothesized phenotypes along with tailored solutions for the phenotypes.

TABLE 3

| Phenotype | Description | Tailored solution |
|---|---|---|
| 1a | Postpartum: GABA delta receptor expression | Allopregnanolone/soyasaponin mixed |
| | does not sufficiently rebound after delivery | self-assembly |
| 1b | Antenatal: GABA delta receptor expression is | Honokiol/Ellagic acid mixed self- |
| | too stunted during pregnancy | assembly |
| 2 | Postpartum: Failure to modulate estrogen levels | Equol/soyasaponin mixed |
| | after delivery | self-assembly |
| 3 | Postpartum: without wishing to be tied to a | Zinc chelated oxytocin micelle |
| | theory, it is believed that oxytocin levels do not | |
| | increase postpartum with a corresponding | |
| | decrease in cortisol as desired. It is | |
| | hypothesized that the elevated cortisol level | |
| | blocks the benefits of oxytocin, so one solution | |
| | is to lower stress levels to allow oxytocin to | |
| | work | |
| 4a | Antenatal: Underlying inflammation leads to | Concentrated |
| | pain, poor sleep, anxiety and rumination | 17-OH DHA |
| | Gestational diabetes mellitus is often present | 17-OH DHA is also effective to prevent |
| | along with the antenatal depression phenotype | or manage gestational diabetes mellitus |
| 4h | Postpartum inflammation stemming from a | Treat with a chelated Trkb-agonizing |
| | complicated delivery does not resolve, thereby | hydroxyflavone and/or a Trkb- |
| | leading to pain, poor sleep, anxiety and | agonizing mixed self-assembly |
| | rumination, but more acutely and intensely than | |
| | phenotype 4a | |

Allopregnanolone/Soyasaponin Mixed Self-Assembly:

Recently, Sage Pharmaceuticals announced very positive results for their phase II trial of intravenous allopregnanolone (SG-547) for postpartum depressed (PPD) mothers. Although encouraging, the requirement of an intravenous administration makes the Sage treatment inconvenient at best and likely subject to frequent noncompliance.

We have developed a treatment involving a mixed micelle of allopregnanolone and soyasaponin. Soyasaponin, which is present in soy infant formula (Fonseca, Food Chem. 2014 Jan. 15; 143:492-8) and so has a demonstrated safety profile, is also a surfactant capable of forming the superstructure of a micelle (DeCroos, Food Chemsitry 101, 2007, 324-323). We have made the novel observation that allopregnanolone and soyasaponin share common structure. Because of this commonality of structure, it is believed that allopregnanolone will intercalate into the soyasaponin superstructure. This intercalation will produce enhanced bonding between the allopregnanolone and the soyasaponin superstructure, and thereby cause allopregnanolone to release from the micelle at a very slow rate that will allow for once-a-day administration.

Honokiol/Ellagic Acid Mixed Self-Assembly:

One hypothesis of postpartum depression (PPD) is that the mother's GABA delta receptors in her brain fail to rebound after birth (Maguire, Pyschoneuroendocrinology, 2009, December 34(Suppl) S84-S90), and that the subsequent PPD can be ameliorated with the administration of a GABA delta agonist (Maguire, Neuron, 2008 Jul. 31, 59(2) 207-213). We have developed a treatment for this phenotype involving an ellagic acid self-assembly intercalated with honokiol. Honokiol, which is present in magnolia bark and has been proposed as a treatment for infant pain (Woodbury, J Nat Prod. 2015 Nov. 25; 78(11):2531-6), is a highly potent GABA delta agonist that increases in vitro GABA currents about 900% at 10 μM (Alexeev, Neuropharmacology, 2012 June, 62(8), 2507-2514). Ellagic acid, which is present in strawberries and has been declared to be GRAS by the FDA, is a biphenyl structure that can form strong pi-pi bonds with other ellagic acid molecules (Frayne, Mater. Express, 2, 4, 2012, 335-343) and so can self-assemble (Barnaby, J. Nanosci. Nanotechnol. 2011 September, 11(9), 7579-86). We have made the novel observation that honokiol and ellagic share a common biphenyl structure having a plurality of hydroxyls extending therefrom. Because of this commonality, it is believed that honokiol will intercalate into the ellagic acid superstructure and thereby release from the ellagic acid self-assembly at a very slow rate.

Equol/Soyasaponin Mixed Self-Assembly:

It has been reported that DNA methylation associated with PPD risk correlated significantly with estrogen-induced DNA methylation change, suggesting an enhanced sensitivity to estrogen-based DNA methylation reprogramming exists in those at risk for PPD (Guintivano, Mol. Psychiatry, 2014 May; 19(5):560-7), and further suggesting that estrogen can be therapeutic for some mothers with this PPD phenotype. However, concern for possible cancer-related side effects of estrogen has stunted its use. The isoflavanol Equol is a soy metabolite that is selective for the beta estrogen (non-cancer) receptor (ERβ) (Sareddy, Chin. J. Nat. Med., 2015 November; 13(11):801-7), and so does not carry a cancer risk. Equol is produced by the ingestion of soy formula, and is thought to have an excellent safety profile. We have made the novel observation that equol and soyasaponin share common structure, and so it is surmised that equol will intercalate into the soyasaponin superstructure and thereby release from the micelle at a very slow rate.

Zinc-Chelated Oxytocin:

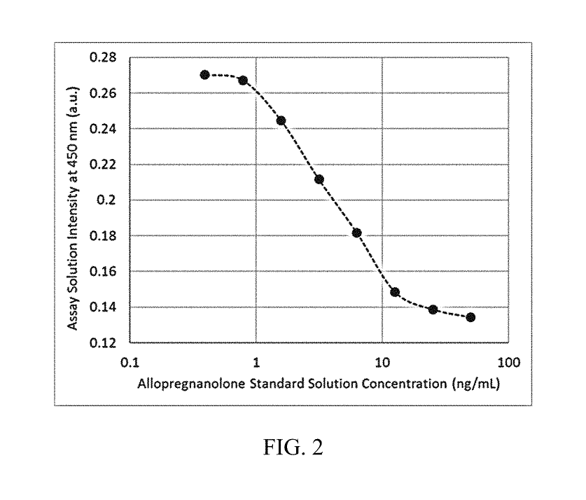

Although some studies report the benefits of oxytocin for PPD mothers, its failure to cross the blood brain barrier (Chapman, Pharm Res. 2013 October; 30(10):2475-84) (thereby requiring an intranasal route of administration), and its short (˜6.8 minute) half-life (Paccamonti, Equine Vet J. 1999 July; 31(4):285-8), thereby requiring multiple dosings per day, prevent its more extensive use. Although oxytocin is not considered to be amphiphilic, the novel observation that several journal articles show diagrams of zinc-chelated oxytocin appearing to have a surfactant-like distribution of hydrophilic and hydrophobic sites is made herein. These articles include: (a) Wyttenbach, J. Am. Chem. Soc., 2008, 130, 5993-6000. Note in FIG. 9c of Wyttenbach the clustering of the Ile, Tyr, Leu and Pro hydrophobic residues in the upper part of the chelate and the clustering of the Asn and Gln hydrophilic residues in the lower part of the chelate; (b) Fuller, J. Am. Soc. Mass Spectrom., 2016, 27, 1376-82. Note in FIG. 4 of Fuller the clustering of the Pro, Leu, Tyr and Ile hydrophobic residues around the lower right part of the figure, and the clustering of the hydrophilic Glu and Asn residues in the upper left part of the figure; and (c) Liu, J. Am. Chem. Soc., 2005, 127, 7, 2024-5. Note in FIG. 2c of Liu the clustering of the Ile, Tyr and Leu hydrophobic residues around the upper right part of the figure and the clustering of the Glu, Asn and Gly-NH₂ residues around the lower left part of the figure.

Moreover, it has been reported that this zinc-chelated oxytocin binds better to the OXT receptor better than oxytocin itself (Liu, J. Am. Chem. Soc. 2005 Feb. 23; 127(7):2024-5). We have developed a phosphatidylcholine-based sustained release device that exploits the surfactant-like nature of the zinc-chelated oxytocin, in which the zinc-chelated oxytocin intercalates into a standard phosphatidylcholine micelle. Because the zinc-chelated oxytocin has hydrophilic and hydrophobic regions that will respectively bond to the hydrophilic and hydrophobic parts of the phosphatidylcholine micelle, it will have greatly enhanced bonding to the micelle superstructure and thereby provide a slower release rate therefrom.

In other embodiments, the zinc-chelated oxytocin forms a self-assembled structure selected from the group consisting of a micelle, a liposome or a multi-lamellar vesicle.

Concentrated 17-OH DHA:

17-hydroxy docosahexaenoic acid (17-OH DHA) is a highly lipophilic fish oil metabolite and the metabolic precursor to neuroprotectin (Basselin, J Lipid Res. 2010 May; 51(5):1049-56), which is a potent anti-inflammatory that strongly upregulates bcl-2 in neurons (Bazan, J. Lipid Research, 51, 2010, 2018-2031 and Mukherjee, PNAS USA. 2004 Jun. 1; 101(22)). Because bcl-2 upregulation is thought to enhance synaptic plasticity (Manji, Biol Psychiatry. 2003 Apr. 15; 53(8):707-42) and inflammation is one biomarker of one antenatal phenotype of PND (Roomruangwong, Mol. Neurobiol., 2016 Feb. 5), administering 17-OH DHA to an antenatally depressed mother should be beneficial towards alleviating that expectant mother's antenatal depression. Moreover, rat studies have shown 17-OH DHA to alleviate the symptoms of diabetes (Neuhofer, Diabetes. 2013 June; 62(6):1945-56). Lastly, 17-OH DHA has been found in mother's milk (Weiss, Lipids Health Disease, 2013, 12, 89) thereby verifying its safety. Therefore, 17-OH DHA looks to be an excellent candidate for administration to an antenatally depressed mother, especially one who suffers from gestational diabetes.

A. Zinc 17-OH DHA Chelate:

We have developed a novel process for concentrating 17-OH DHA from cow's milk. First, we start with the widely-available milk fat fraction that is a byproduct of the production of skim milk. Next, we have made the novel observation that 17-OH DHA has great structural similarity to ricinoleic acid, and we believe that the chelated complex zinc ricinoleate has enhanced water solubility. Accordingly, we believe 17-OH DHA will form a chelate with zinc substantially in the same way that ricinoleic acid forms a chelate with zinc and that the chelated 17-OH DHA complex will likewise have an enhanced water solubility. We thus propose to concentrate 17-OH DHA from the fat fraction by its zinc chelation and subsequent movement of the chelate into an aqueous phase. The novel components of the resulting aqueous phase (which likely also contains anti-inflammatory, chelatable resolvins and lipoxins) can be marketed as a 17-OH DHA-rich product.

B. Cyclodextrin—17-OH DHA Complex:

There is provided a second novel process for concentrating 17-OH DHA from cow's milk. This method begins with the widely-available milk fat fraction that is a byproduct of the production of skim milk. In other embodiments, the starting fluid is a marine oil such as fish oil, krill oil or algae oil. In other embodiments, the starting fluid is an algae-derived oil. Next, it is observed that the melting points of long chain fatty acids are substantially controlled by the number of cis-double bonds in the molecule, as shown in Table 4. Because 17-OH DHA has five double bonds, it likely has one of the lowest melting points of the fatty acids in milk fat. This feature can be exploited to concentrate 17-OH DHA in milk fat. Accordingly, in one embodiment, the milk fat fraction is subject to selective freezing in a temperature range of about at −20° C. to −40° C., thereby separating the lowest melting point molecules (i.e., those fatty acids having 4-6 cis bonds) from the remainder of the fat fraction. It is believed that this step removes about 95% of the fatty components in the milk fat fraction, and so concentrates 17-OH DHA by a factor of about 19.

TABLE 4

| | Cis | | Melting Point |
|---|---|---|---|
| Constituent | Double Bonds | Concentration | (° C.) |
| Palmitic Acid | 0 | 23.96% | 64 |
| Stearic Acid | 0 | 6.91% | 70 |
| Oleic Acid | 1 | 46.29% | 13 |
| Palmitoleic Acid | 1 | 3.35% | 1 |
| Linoleic Acid | 2 | 14.42% | −5 |
| Gamma-linolenic Acid | 3 | 0.09% | −11 |
| Alpha-linolenic Acid | 3 | 1.07% | −12 |
| Arachidonic Acid | 4 | 2.09% | −49 |
| Docosahexanoic Acid | 6 | 1.15% | −44 |
| Eicosapentaenoic Acid | 5 | 0.08% | −65 |
| Lipoxin A4 | 1 | 15.55 ng/ml | ? |
| Resolvin E1 | 2 | 4.24 ng/ml | ? |
| Re solvin D1 | 3 | 9.42 ng/ml | ? |
| 17-OH DHA | 5 | 53.38 ng/ml | ? |

Weiss et al., Lipids Health Disease 12: 89 (2013), Tables 1-3

Next, we make the observation that monohydroxyfatty species are rapidly taken up by cells and esterified into triglycerides (Stenson, Prostaglandins, 1983, August, 26(2) 253-64, and Brezinski, PNAS USA, 1990 August, 87(16) 6248-52), and because adipose cells are extensively present in mammaries, it may reasonably be concluded that the fatty acids in breastmilk are also present in the triglyceride form. We then observe that hydroxyfatty acids can be selectively released from triglycerides by PLA2 without releasing the nonhydroxylated fatty acid species from the triglycerides (van Kuijk, Trends in Biochem. Sci., 12, 1987, 31-34; Bayon, Plant Physiology, April 2015, 167, 1259-70; and Bafor, Biochem. J., 1991, 280, 507-514). Accordingly, we can selectively release hydroxyfatty acids from its parent triglyceride by contacting the low MP milk fat fraction against immobilized PLA2.

Once the hydroxyfatty acids (and 17-OH DHA in particular) are present in their free form, they can be selectively removed from solution by contacting the solution with a cyclodextrin. Beta-Cyclodextrin is a lipophilic tube having an inner pore size of about 7 Angstroms (U.S. Pat. No. 4,902,788), and so beta-cyclodextrins can be used to remove/concentrate lipophilic molecules having a size less than 7 Angstroms. Because the cis bond-driven folding of DHA causes it to have a radial dimension of about 5 Angstroms (Yonezawa, Int. J. Mol. Med., 2006, October, 18(4) 583-8 at Table 1), and 17-OH DHA is structurally quite similar, it is reasonable to conclude that 17-OH DHA has a radial dimension of about 7 Angstroms and so can likewise enter into and thereby be concentrated in cyclodextrins. Moreover, the nonhydroxylated fatty acids, such as DHA, remain in triglyceride form. Because there are 3 DHA molecules in a DHA triglyceride, the dimensions of the triglyceride are probably about 15 Angstroms, and so would be much too large to be captured by beta-cyclodextrin. Accordingly, the cyclodextrins can selectively concentrate free 17-OH DHA from triglycerides.

In one embodiment, the cyclodextrin is present as a cyclodextrin carbonate nanoparticle, as described in Zhang, Intl. J. Nanomedicine, 2015, 10, 3291-3302. It is believed that both the cyclodextrin and carbonate components are perfectly safe for infants. Because Zhang's cyclodextrin-carbonate nanoparticle has a pore size of about 136-242 Angstroms, 17-OH DHA (which has a 5 Angstrom dimension) can easily diffuse through its pore system to be ultimately captured by the cyclodextrin. Moreover, cyclodextrin carbonate nanoparticles can be used to build a filter column through which the free hydroxylated fatty acid solution can be passed. Once in the cyclodextrin carbonate nanoparticle column, the free hydroxylated fatty acids (including 17-OH DHA) will enter the pore of the cyclodextrin and be captured thereby.

In another embodiment, the cyclodextrin is replaced by zeolite. Zeolite has been ingested for centuries by pregnant women in the form of clay to capture the nutritional mineral content of clay, relieve vomiting and nausea, and protect the digestive tract. Zeolite has a pore size of about 10 Angstroms (Du, J. Physics Chem. Solids, 68(2007) 1692-99), and so zeolites can be used to remove/concentrate molecules having a size less than 10 Angstroms. Because the cis bond driven folding of DHA causes it to have a radial dimension of about 5 Angstroms (Yonezawa, Int. J. Mol. Med., 2006, October, 18(4) 583-8 at Table 1), and 17-OH DHA is structurally quite similar, it is reasonable to conclude that 17-OH DHA can likewise be concentrated in zeolite.

In another embodiment, the cyclodextrin is replaced by mesoporous silica. Mesoporous materials have a pore size of 20-500 angstroms (Wikipedia), and so they can be used to remove/concentrate lipophilic molecules having a size less than 50-300 angstroms. Because DHA has a 5 angstrom size, and 17-OH DHA is structurally quite similar, it is reasonable to conclude that 17-OH DHA can likewise be concentrated in mesoporous silica.

In another embodiment, the cyclodextrin is replaced by octadecyl silyl silica (OSS). OSS has been used to selectively remove/concentrate hydroxylated molecules (i.e., prostaglandins) from phospholipids (Powell, Prostaglandins, 1980, November, 20(5) 947-57). Because 17-OH DHA is likewise hydroxylated, it is reasonable to conclude that 17-OH DHA can likewise be concentrated in OSS.

In sum, a 17-OH DHA concentrated fraction of milk can be made by (a) removing the fat fraction from milk, (b) freezing the fat fraction at about −30° C. to separate out the low melting point fatty acids from the higher melting point fatty acids, (c) contacting the low melting point molecules with immobilized PLA2 to selectively free the hydroxyfatty acids from the triglycerides, and (d) contacting the free hydroxyfatty acids with cyclodextrins (or a substitute described above) to selectively capture the free hydroxyfatty acids without capturing the triglycerides.

The 17-OH DHA/cyclodextrin complex can then be orally administered to the patient, wherein the 17-OH DHA is slowly released by the cyclodextrin.

Therefore, there is provided a method of making a concentrated hydroxyfatty acid fraction from a DHA-containing fluid comprising i) low melting point fatty acids and ii) high melting point fatty acids, wherein both acids are present in triglycerides, comprising the steps of (a) freezing the fluid to separate out the low melting point fatty acids from the high melting point fatty acids contained therein and thereby produce a low melting point-enriched fluid comprising parent triglycerides, (b) ex vivo contacting the low melting point-enriched fluid with PLA2 to selectively free hydroxyfatty acids from their triglycerides to produce a free hydroxyfatty acid-enriched fluid, and (c) ex vivo contacting the free hydroxyfatty acid-enriched fluid with a concentrator to selectively capture the freed hydroxyfatty acids within the concentrator to produce a concentrator having hydroxyfatty acid adsorbed thereon.

Preferably, the freezing step is carried out at about −20° C. to −40° C. The starting fluid can be selected from the group consisting of a marine oil, an algae oil, and a milk. In some embodiments, a step of separating out a fat fraction of the milk is carried out prior to step a), and step b) is carried out on the separated fat fraction.

In some embodiments, the concentrator is present as a cyclodextrin carbonate nanoparticle. In others, the concentrator is selected from the group consisting of cyclodextrin, zeolite, mesoporous silica, and octadecyl silyl silica (OSS).

In some embodiments, the method further comprises the step of (d) administering the concentrator having hydroxyfatty acid adsorbed thereon to a human.

In some embodiments, the concentrator having hydroxyfatty acid adsorbed thereon is enriched in adsorbed 17-OH DHA.

In some embodiments, the concentrator is a solid porous body, and the hydroxyfatty acid is adsorbed within the porosity of the concentrator.

In some embodiments, the PLA2 is immobilized PLA2.

In some embodiments, the method further comprises the steps of (d) releasing the hydroxyfatty acid from the concentrator to produce a hydroxyfatty acid-enriched solution, and (e) administering the hydroxyfatty acid-enriched solution to a human.

There is also provided a method of making a concentrated hydroxyfatty acid fraction from a DHA-containing fluid comprising triglycerides comprising hydroxyfatty acids, comprising the steps of (a) ex vivo contacting the fluid with PLA2 to selectively free hydroxyfatty acids from their triglycerides and thereby produce a free hydroxyfatty acid-enriched fluid, and (b) ex vivo contacting the free hydroxyfatty acid-enriched fluid with a concentrator to selectively capture the freed hydroxyfatty acids within the concentrator to produce a concentrator having hydroxyfatty acid adsorbed thereon. This method can further comprise the step of (c) administering the concentrator having hydroxyfatty acid adsorbed thereon to a human.

This method can alternatively further comprise the steps of (c) releasing the hydroxyfatty acid from the concentrator to produce a hydroxyfatty acid-enriched solution, and (d) administering the hydroxyfatty acid-enriched solution to a human.

In some embodiments, the concentrator having hydroxyfatty acid adsorbed thereon is selectively removed from the free hydroxyfatty acid-enriched fluid.

In some embodiments, the method can further comprise separating the concentrator having hydroxyfatty acid adsorbed thereon from the fluid.

In some embodiments, the method further comprises the step of (c) administering the separated concentrator having hydroxyfatty acid adsorbed thereon to a human, wherein the administration is carried out once a day.

There is also provided a method of making a concentrated hydroxyfatty acid fraction from a DHA-containing fluid comprising esters comprising hydroxyfatty acids, comprising the steps of (a) ex vivo contacting the fluid with an enzyme to selectively free hydroxyfatty acids from their esters and thereby produce a free hydroxyfatty acid-enriched fluid, and (b) ex vivo contacting the free hydroxyfatty acid-enriched fluid with a concentrator to selectively capture the freed hydroxyfatty acids within the concentrator to produce a concentrator having hydroxyfatty acid adsorbed thereon.

Chelated Magnolol Complex:

Magnolol is an isomer of honokiol and is also derived from magnolia bark. In addition, magnolol is likewise a GABA delta agonist, increasing GABA currents by about 900% at a concentration of 10 uM (Alexeev, Neuropharmacology, 2012 June, 62(8), 2507-2514). Therefore, magnolol is deemed to be a suitable replacement for honokiol in the honokiol/ellagic acid assembly discussed above.

Further, examination of the magnolol structure reveals another interesting characteristic of magnolol. In particular, it has been observed that if the phenyl rings of magnolol are properly rotated, the two hydroxyl groups situated on the different rings approach each other. When the hydroxyls are in this “close approach” conformation, it is believed that magnolol can form a chelated complex with metal ions.

The chelated magnolol complex is interesting from a number of viewpoints. First, as opposed to the poor water solubility of pure magnolol, the chelated magnolol complex is likely highly water soluble. The high water solubility of the chelated magnolol complex facilitates the ability of magnolol to approach the epithelial cells in the GI tract by providing high dispersability, and thus increasing its bioavailability in oral administration.

Chelated 7,8 Dihydroxyflavone Complex:

7,8 dihydroxyflavone (7,8 DHF) is a natural flavonoid found in Godmania aesculifolia, Tridax procumbens, and primula tree leaves. It is also available as supplement. Liu reported that 7,8-DHF (which is orally available and BBB penetrable) can specifically activate TrkB receptors (at a low concentration of 250 nM) and its downstream PI3K/Akt and MAPK receptors in the mouse hippocampus. 7,8-DHF can protect neurons from excitotoxic and oxidative stress-induced apoptosis and cell death. Moreover, 7,8-DHF promotes the survival and reduces apoptosis in cortical neurons of traumatic brain injury (Liu, Trans. Neurodegener. 2016; 5: 2). Accordingly, 7,8 DHF is of interest as an antidepressant.

Inspection of the 7,8 DHF molecule reveals adjacent hydroxyl groups attached to an aromatic ring. It is known that such structures have the ability to form chelation complexes with metals.

It is believed that a chelate complex of 7,8 DHF will have high water solubility, thus allowing for its uniform dispersal in the aqueous phase of the gastrointestinal tract, and thereby increasing its bioavailability. Once the well-dispersed 7,8 DHF chelate complex enters the stomach, the high acid content therein will release the metal ion from its complex with 7,8 DHF, leaving a well dispersed, free 7,8 DHF in solution.

Also according to Liu, it is further known that 7,8,2′ DHF and 7,8,3′ trihydroxyflavones (THF) are also potent Trkb agonists, increasing Akt phosphorylation over 150%. Liu, J. Med. Chem, 2010 Dec. 9; 53(23):8274-86. Because these molecules also possess the structure of adjacent hydroxyl groups attached to an aromatic ring, it is likewise believed that such structures have the ability to form chelation complexes with metals. Therefore, it is believed that a chelate complex of 7,8 DHF; 7,8,2′ THF or 7,8,3′ THF (along with other TrkB-active hydroxyflavones having hydroxyls in both the 6 and 7 positions) will have high water solubility, thus allowing for its uniform dispersal in the aqueous phase of the gastrointestinal tract, and thereby increasing its bioavailability.

Once the well-dispersed hydroxyflavonoid chelate complex enters the stomach, the high acid content therein will release the metal ion from its complex with hydroxyflavonoid, leaving a well dispersed, free hydroxyflavonoid in solution.

As these hydroxyflavones having hydroxyls in either the 6,7 or 7,8 positions have been shown to be Trkb agonists, and TrkB is the prime receptor for BDNF, it is believed that these phytochemicals would be good treatment candidates for mothers diagnosed with PND who have low serum BDNF levels, as the phytochemical would serve to augment the heretofore insufficient serum BDNF level in activating the TrkB receptor. Moreover, it has been reported that there is an association between low serum BDNF levels in early pregnancy and antenatal depression (Fung, BMC Psychiatry, 2015 Mar. 10, 15, 43). Therefore, it is believed that these chelates would be good candidates for mothers who have low serum BDNF levels in early pregnancy, so as to prevent the onset of antenatal depression in these mothers.

c. 7,8 Dihydroxyflavone/Hesperetin Glucuronide Mixed Self-Assembly:

Hesperidin is a natural flavonoid glycoside found in citrus. Its deglycosylated flavonoid metabolite (hesperetin) is commonly found in significant quantities (up to micromolar levels) in human mother's milk (Song, Nutrition, 2013 January; 29(1):195-202). Hesperidin administration is further known to increase BDNF levels in chronically-depressed rats (Donato, Brain Res. Bull., 2014 May; 104:19-26). Moreover, its metabolite hesperetin induces BDNF (Hwang, J. Agric. Food Chem., 2011 May 25; 59(10):5779-85). Another metabolite, hesperetin glucuronide, has a log P of 0.12 (Chemspider), and so, it is reasonably assumed that hesperetin glucuronide will behave like a surfactant, and thereby have the ability to form micelles, liposomes and MLVs.

It is further observed that the flavanone hesperetin glucuronide shares the same basic flavonoid structure (with the exception of a double bond) as the flavonol 7,8 DHF. Therefore, it is believed that 7,8 DHF might intercalate within a hesperetin glucuronide self-assembly to form a mixed structure that will slowly release 7,8 DHF therefrom.

This same hesperetin glucuronide molecule has been found to activate PPAR-gamma receptors (EC˜100 uM) and so likely has utility as an anti-diabetic agent (Gamo, Chem. Pharm. Bull., 62(5), 491-493 (2014)). Therefore, it is believed that this 7,8 Dihydroxyflavone/Hesperetin Glucuronide mixed self-assembly can be useful for treating the Group 4a phenotype described above.

d. Hesperetin/Hesperetin Glucuronide Mixed Self-Assembly:

As discussed above, it is known that hesperetin is found in significant quantities in mother's milk and is known to induce BDNF. Therefore, it is an attractive candidate for use as an antidepressant in PND. However, its high lipophilicity (Log P=2.90) causes it to have a low dispersability in water and therefore a low bioavailability. It is further observed that hesperetin shares the identical flavonoid structure as its metabolite, hesperetin glucuronide. As discussed above, the amphiphilic log P of hesperetin glucuronide leads one to believe that it can self-assemble. Therefore, it is believed that hesperetin might intercalate within a hesperetin glucuronide self-assembly to form a mixed structure that will be well dispersed in water (and therefore have a high bioavailability) and will also likely provide for slow release of hesperetin therefrom.

e. Pinocembrin/Hesperidin Glucuronide Mixed Self-Assembly:

Pinocembrin, the primary flavonoid in Swiss honey, has been reported to suppress apoptosis in neurons with an EC₅₀ of only 100 nM (Jang, PNAS, 107, 6, 2687-92). It is further observed that hesperidin glucuronide shares the identical flavanone structure as pinocembrin. Therefore, it is believed that pinocembrin can intercalate within a hesperidin glucuronide self-assembly to form a mixed structure that will slowly release pinocembrin therefrom.

Likewise, owing to their structural similarity, it is believed that pinocembrin can intercalate within a saponin self-assembly (such as a soyasaponin self-assembly) to form a mixed structure that will slowly release pinocembrin therefrom.

f. 7,8 Dihydroxyflavone/7,8 Dihydroxyflavone Glucuronide Mixed Self-Assembly:

As discussed above, 7,8 dihydroxyflavone is an attractive candidate as a PND anti-depressant because it is a potent TrkB agonist. It is now observed that its metabolite, 7,8 dihydroxyflavone glucuronide, shares common structure with hesperetin glucuronide and so likely has an amphiphilic log P similar to hesperetin glucuronide. This amphiphilic property would make 7,8 dihydroxyflavone glucuronide a good candidate for self-assembly.

It is further observed that 7,8 dihydroxyflavone glucuronide shares the identical flavonoid structure as 7,8 dihydroxyflavone. Therefore, it is believed that 7,8 dihydroxyflavone can intercalate within a 7,8 dihydroxyflavone glucuronide self-assembly to form a mixed structure that will provide for high bioavailability of 7,8 dihydroxyflavone and slowly release 7,8 dihydroxyflavone therefrom.

As with the above chelates, it is believed that these self-assemblies that augment BDNF or activate TrkB would be good candidates for expectant mothers who have low serum BDNF levels in early pregnancy, so as to prevent the onset of antenatal depression in these mothers.

g. Hyperoside/G-Rutin (SJW) Mixed Self-Assembly:

St. John's Wort (SJW) is one of the few antidepressant preparations available to a nursing mother diagnosed with PND for which there is evidence demonstrating both safety and efficacy. It has been reported that in vivo rat experiments demonstrate that the hyperoside (0.6 mg/kg) and quercetin-3-glucuronide (0.6 mg/kg) constituents in SJW appear (along with hyperforin) to be the entities responsible for the anti-depressant effect of SJW, and that they work by reducing the HPA axis function by reducing plasma levels of ACTH and cortisol by 40-70% (Butterweck, Planta Med., 2000, February, 66(1) 3-6). As there are some concerns with SJW and its side effects, in particular a deleterious interaction between the hyperforin in SJW and blood pressure medication (Narhstedt, J. Nat. Prod., May 2010, 73(5) 1015-21), it would appear that providing just the beneficial components of SJW to the PND mother seems like an attractive option. In this respect, hyperoside in particular seems to have promise as a PND treatment option, as it has been shown to elevate BDNF transcription in PC12 cells (Zheng, Phytomedicine, 2012, Jan. 15, 19(2) 145-9). Moreover, a hyperoside-rich preparation (Venetron) is commercially available in the US and has been clinically demonstrated to improve symptoms of depression. Maypro, “Venetron: A Promising New and Efficacious Dietary Ingredient for mood support and sleep.”

However, it is further observed that hyperoside is rather lipophilic, possessing a log P of 1.75 (Chemspider). Because of this high lipophilicity, hyperoside is likely not very water soluble and so likely has difficulty in attaining a high oral bioavailability. Moreover, being a polyhydroxyflavone, hyperoside is likely subject to severe first-pass metabolism, thereby reducing its potency. Accordingly, it is a goal to provide hyperoside in a delivery package that increases its bioavailability and release profile.

In order to overcome this oral bioavailability issue, it is proposed that hyperoside be housed in a self-assembly of glycosylated rutin (G-rutin). G-rutin is a natural amphiphilic phytochemical that is found in buckwheat and the Japanese Pagoda tree (Morita, Cereal Chem, 1996, 73(1) 99-104). It is currently marketed in the US as an active ingredient in Eucerin® skin lotions, and has particular interest for consumers with sun allergy. Of interest, G-rutin has been reported to self-assemble into micellar aggregates (Tozuka, Eur. J. Pharm. Biopharm., 2012 September; 82(1):120-6). These micelles should have a high water solubility, and so should have a high oral bioavailability.

It is further appreciated that hyperoside and G-rutin have a special relationship by virtue of their nearly identical structures. In particular, each of hyperoside and G-rutin has a quercetin-based lipophilic portion and glucose moieties attaching off the same 3-OH of the base quercetin molecule.

Because of the near identity in their chemical configurations, it is believed that hyperoside will neatly intercalcalate itself within the G-rutin superstructure of the micelle. This neat intercalation will result in enhanced bonding between the lipophilic portions of the quercetin base molecules, between the hydroxyls of the base quercetin molecules, and between the glucose structures that attach to the 3-OH portion of the quercetin molecules. This enhanced bonding will likely result in an extended time of release of the hyperoside from the self-assembly, which can allow for a much longer hyperoside half-life in circulation, thereby increasing the bioavailability of hyperoside. Accordingly, it can be appropriate to consider the hyperoside/G-rutin mixed self-assembly as a phytosome.

In some embodiments, the hyperoside/G-rutin self-assembly manifests itself as a micelle. In others, the hyperoside/G-rutin mixed self-assembly manifests itself as a liposome. In others, the hyperoside/G-rutin self-assembly manifests itself as a multi-lamellar vesicle (MLV).

h. Q3G/G-Rutin Mixed Self-Assembly:

It is further observed that Q3G is rather lipophilic, possessing a log P of 2.10 (Chemspider). Because of this high lipoiphilicity, Q3G is not very water soluble and so likely has difficulty in attaining a high oral bioavailability. Moreover, the plasma elimination half-life of Q3G has been reported to be only 2.33 hours (Mullen, Br. J. Nutr. 2006 July; 96(1):107-16). Mullen further reported that the profile of metabolites excreted in urine was markedly different to that of plasma with many of the major urinary components, including quercetin-3′-glucuronide, two quercetin glucoside sulphates and a methylquercetin diglucuronide, absent or present in only trace amounts in the bloodstream, indicative of substantial phase II metabolism.

Therefore, it is a goal of the present invention to increase the bioavailability and half-life of Q3G.

In order to overcome this oral bioavailability issue, it is proposed that Q3G be housed in a self-assembly of glycosylated rutin (G-rutin). G-rutin is an amphiphilic molecule that has been reported to self-assemble into micellar aggregates (Tozuka, Eur. J. Pharm. Biopharm., 2012 September; 82(1):120-6). These micelles should have a high water solubility, and so should have a high oral bioavailability. Therefore, G-rutin should be able to sufficiently deliver Q3G from the GI tract into the circulatory system.

It is further appreciated that Q3G and G-rutin have a special relationship by virtue of their highly similar structures. In particular, each of Q3G and G-rutin has a quercetin-based lipophilic portion and a glucose-like portion attaching off the same 3-OH of the base quercetin molecule.

Because of the near identity in their chemical configurations, it is believed that Q3G will neatly intercalate itself within the G-rutin superstructure of the micelle. This neat intercalation will result in enhanced bonding between the lipophilic portions of the quercetin base molecules, between the hydroxyls of the base quercetin molecules, and between the glucose-like structures that attach to the 3-OH portion of the quercetin molecules. This enhanced bonding will likely result in an extended time of release of the Q3G from the self-assembly, which can allow for a much longer Q3G half-life in circulation, thereby increasing the bioavailability of Q3G. Accordingly, it can be appropriate to consider the Q3G/G-rutin assembly as a phytosome.

In some embodiments, the G-rutin/Q3G self-assembly manifests itself as a micelle. In others, the G-rutin/Q3G self-assembly manifests itself as a liposome. In others, the G-rutin/Q3G self-assembly manifests itself as a multi-lamellar vesicle (MLV).

As both hyperoside and Q3G appear to promote antidepressant state by reducing ACTH and cortisol levels (see Butterweck above), and high cortisol levels are implicated in the phenotype 3 presented above, it appears that the hyperoside/G-rutin self-assembly and the Q3G/G-rutin self-assembly presented herein would have special applicability to the mother diagnosed with the above Type 3 phenotype.

i. Ginsensoide Liposomes and MLVs:

The ginsenoside Rg3 has been reported to exert antidepressant effects in several animal models (Cui, J. Psychopharmacol., 2012 May; 26(5) 697-713). Investigators have further reported the ginsenoside Rg2 reverses stress-induced depression-like behaviours and BDNF expression within the prefrontal cortex (Zhu X, Eur J Neurosci. 2016 July; 44(2):1878-85); the beneficial effects of ginsenoside Rg1 on chronic stress-induced depression-like behaviours, BDNF expression and phosphorylation of PKA and CREB, (Liu Z, Neuroscience. 2016 May 13; 322:358-69); the beneficial effect of ginsenoside Re on depression and anxiety-like behaviours induced by repeated immobilization (Lee B, J Microbiol Biotechnol. 2012 May; 22(5):708-20); and the antidepressant effects of ginsenoside Rg1 due to activation of BDNF signaling and neurogenesis in the hippocampus (Jiang, Br J Pharmacol. 2012 July; 166(6):1872-87).

Therefore, in some embodiments, ginsenoside self-assembled structures (and in particular liposomes and MLVs) are administered to the mother diagnosed with either antenatal or postnatal PND.

As discussed above, it has been reported that ginsenosides self-assemble into micelles, and that self-assembled ginsenoside micelles can be tuned to have release rates from days to months. Xiong, Int. J. Pharm. 2008 Aug. 6; 360(1-2):191-6. The tuning is performed by varying the concentration of the ginsenoside in the initial solution, with higher concentrations leading to slower release rates. Xiong discloses that loading determines release rate. In some embodiments, the loading of the ginsenoside self-assembly (and in particular Rg3) is targeted to provide a release rate that corresponds to essentially complete release in about 24 hours and a 50% release rate at about 12 hours. This loading and corresponding rate would enable the mother to take only one dose a day (and thereby promote compliance more than a multiple-dose-per-day routine) while still enabling a habit-forming routine of taking one dose per day (thereby promote compliance more than a one-dose-every-few-days routine).

In some embodiments, the ginsenoside self-assembly (and in particular, the Rg3 self-assembly) is presented in the form of liposomes. It is believed that the liposomal form provides an advantage during pulmonary administration of the the ginsenoside self-assembly (and in particular, the Rg3 self-assembly). Liposomes are generally on the order of 100-200 nanometers (and so are categorized as fine particles), while micelles are much smaller at about 10-20 nm (and so are categorized as ultrafine particles). Because a substantially larger fraction of micelles are exhaled after pulmonary administration, liposomes provide an advantage over micelles in that their relatively larger size provides a much more efficient pulmonary administration. Liposomes can also deliver hydrophilic molecules housed in their aqueous cores.

In some embodiments, the ginsenoside self-assembly (and in particular, the Rg3 self-assembly) is presented in the form of multi-lamellar vesicles (MLVs). It is believed that the MLV form provides an advantage during pulmonary administration of the ginsenoside self-assembly (and in particular, the Rg3 self-assembly). MLVs can be made to a size on the order of a few microns. Because a substantially larger fraction of micelles and liposomes are exhaled after pulmonary administration, MLVs provide an advantage over micelles and liposomes in that the relatively larger size of MLVs provides a much more efficient pulmonary administration. Thus, MLVs have the particle diameter in the range needed for aerosol delivery to the alveolar region (Zaru, Eur. J. Pharmaceutics Biopharm., 67(2007) 655-666 at 663). These ginsenoside inventions will have a slower release than conventional drug-loaded liposomes because the drug forms part of the superstructure of the micelle/liposome/MLV and so has enhanced intermolecular bonding.

In sum, the above disclosure provided a number of broad concepts involving matching self-assembling amphiphilics with active agents capable of intercalating within the self-assembly. Some of these examples are listed in Table 5.

TABLE 5

| | | | Amphiphilic |
|---|---|---|---|
| Active Class | Active Example | Amphiphilic Class | Example |
| Flavone | 7,8 dihydroxy- | Flavone-7-O- | 7,8 dihydroxy- |
| | flavone | glucuronide | flavone-7-O- |
| | | | glucuronide |
| Flavone-3-O- | Miquelianin; | Flavone-3-O- | G-Rutin |
| glucuronide | | glycoside | |
| Flavone-3-O- | Hyperoside | Flavone-3-O- | G-Rutin |
| glycoside | | glycoside | |
| Flavanone | Hesperetin; | Saponin | soyasaponin; |
| | pinocembrin | | quillaja |
| Flavone | 7,8 dihydroxy- | Flavone-3-O- | G-Rutin |
| | flavone | glycoside | |
| Isoflavone | Equol | saponin | soyasaponin; |
| | | | quillaja |
| Isoflavone | Equol | Isoflavone glycoside | diadzin |
| Steroid | allopregnanolone | saponin | soyasaponin; |
| | | | quillaja |
| Flavanone | Hesperetin; | Flavanone-7-O- | Hesperetin-7-O- |
| | pinocembrin | glucuronide | glucuronide |

Thus, generally, there is provided herein a mixed self-assembly comprising (a) a hydroxylated flavonoid-7-O-glucuronide self-assembly (preferably a hydroxylated flavanone-7-O-glucuronide self-assembly), and (b) a hydroxylated flavonoid (preferably a hydroxylated flavanone) intercalated within the self-assembly.

Thus, generally, there is provided herein a mixed self-assembly comprising (a) a hydroxylated flavonoid-3-O-glycosyl self-assembly (preferably a hydroxylated flavonol-3-O-glycosyl self-assembly, and (b) a hydroxylated flavonoid (preferably a hydroxylated flavonol) intercalated within the self-assembly.

Thus, generally, there is provided herein a hydroxyflavone having adjacent hydroxyl groups that is chelated by a metal (preferably zinc) that forms a complex with the two hydroxyls. Preferably, the hydroxyflavone chelate forms a self-assembly with other similar complexes.

Baicalin

It is believed that sufficient progesterone is important to the expectant mother in two ways. First, it has been reported that lower progesterone in the second trimester of pregnancy is associated with greater negative emotional responses to stress in that trimester (Crowley, Psychopharmacology, 2016 April, 233(7), 1299-310). Second, it has been repeatedly reported that prophylactic administration of progesterone can reduce the incidence of preterm births (Saccone, Ultrasound Obstet Gynecol., 2016 Aug. 22). It is believed that preterm birth is positively associated with symptoms of PND. For example, mothers of early, moderate, and late preterm infants reported similar rates of possible depression (20%, 22%, and 18%, respectively) one month after NICU discharge (Hawes, J. Pediatr. 2016 Aug. 5. pii: S0022-3476(16)30531-5). These depression rates associated with preterm births are somewhat higher than the 10-15% PPD rate generally reported. Moreover, another investigator group reported that premature infants at three months exhibit more withdrawal behavior and their mothers reported elevated maternal depressive symptoms as compared with the full-born group. At 12 months, the mothers of the premature infants reported more child internalizing behavior (Moe, Infant Behav. Dev., 2016 August; 44:159-68). Therefore, it appears that properly provided progesterone can be helpful to the expectant mother having low progesterone levels or at risk for delivering preterm. However, progesterone has a relatively short circulatory half life (Anand Kumar. Proc. Nat. Acad. Sci. USA, 1982 July; 79(13):4185-9). Therefore, therapies that help increase the circulatory half-life of progesterone can be useful.

Baicalin is a glucuronidated flavonoid found in the Chinese Skullcap extract, which has been used in Chinese medicine for miscarriage and threatened abortion (Chen, Evidence-based Compl. Alter. Med., Volume 2011, 408714). Baicalin can exert antiabortive effects by reducing IFN-gamma levels and elevating progesterone (Ma, Am. J. Chin. Med., 2009, 37(1) 85-95 and Chen, J. Steroid Biochem. Mol. Biol. 2015 May; 149:11-6 (Baicalin elevating progesterone)). In an in vitro study of the effect of baicalin on deidua cells, baicalin showed a nonsignificant trend in elevating progesterone (Wang, J. Immunol. Research, Vol. 2014, 859812, FIG. 6). Baicalin has demonstrated tocolytic properties, meaning it can delay labor, and investigators attribute the tocolytic properties of baicalin to its ability to increase progesterone (Chen, J. Steroid Biochem. Mol. Biol. 2015 May; 149:11-6). Baicalin appears to be a better tocolytic agent than its aglycone bacailein (Chen, citing Ma, Chin. J. Vet. Sci. 27 (2007) 412-415 (in Chinese)).

Therefore, without wishing to be tied to a theory, it appears that baicalin administration to an expectant mother might be useful for increasing the mother's progesterone levels, thereby elevating mood and decreasing the risk of preterm birth.

It is further noted that bacailin is structurally similar to scutellarin (differing by a single hydroxyl), in that each is a hydroxylated flavonoid-7-O-glucuronide. Accordingly, it is reasonable to expect that their pharmacologic profile should be reasonably similar.

It appears that hydroxylated flavonoid-7-O-glucuronides such as scutellarin are poorly orally available, demonstrating a bioavailability of less than 3% (Liu, Eur. J. Pharm. Biopharm., 2008 November, 70(30 845-52). Because of the structural similarity of scutellarin and baicalin, it is reasonable to conclude that baicalin has a similarly poor oral bioavailability. Liu goes on to report, however, that providing spray-dried scutellarin nanoparticles through a pulmonary administration increased the bioavailability of the scutellarin to about 77%, and that adding a mucoadhesive excipient to the formulation increased to bioavailability to over 95%.

Because bacailin is structurally similar to scutellarin, it is reasonable to conclude that likewise providing spray-dried baicalin nanoparticles through a pulmonary administration can increase its bioavailability from a level of less than 3% to about 77%, and that adding a mucoadhesive excipient to the formulation might increase its bioavailability to over 95%.

Therefore, there is provided a method of treating an expectant mother (preferably having a risk of preterm birth), comprising the steps of (a) providing an inhaler housing a formulation comprising spray-dried baicalin nanoparticles and a mucoadhesive excipient, (b) carrying out a pulmonary administration of the formulation to the expectant mother.

Liu, supra, reported that the plasma concentration of scutellarin in their experiments decreased by a factor of about 10 over the course of about an hour, thereby suggesting that scutellarin (and by implication other hydroxylated flavonoid-7-O-glucuronides such as baicalin) has a short circulatory half-life. It has been reported that baicalin has a log P of 1.27 (Liang, J. Agric. Food Chem., 2009 Aug. 12; 57(15):7118-24) and so can be considered to be amphiphilic. Therefore, in preferred embodiments, this amphiphilic quality is exploited to provide baicalin in the form of a self-assembly such as a liposome or MLV that can provide for an extended release of baicalin.

Cerebroside and bcl-2

In some embodiments, an amphipathic cerebroside is made into a self-assembly in the form of a liposome or MLV, and is delivered (preferably by the oral or pulmonary route) to a mother diagnosed with PND. Cerebrosides are endogenous molecules known to be present in human milk (Newburg, Lipids, 1992 November; 27(11):923-7). Certain aquatic cerebrosides have also been reported to dramatically increase the gene expression of B-cell lymphoma 2 (Bcl-2) (Wu, J. Oleo Sci., 2013; 62(9):717-27). Bcl-2 is an anti-apoptotic gene that has been implicated in mediating neuronal plasticity (Manji, Psychopharmacol. Bull., 2001 Spring; 35(2):5-49). Therefore, it is expected that administration of a cerebroside to a mother diagnosed with PND should increase her neuronal plasticity and thereby alleviate her symptoms of depression.

Cancer Applications

In addition to applications of phytochemical self-assemblies directed to maternal depression, it is believed these phytochemical self-assemblies can also be directed to certain forms of cancer.

a. Hyperoside and Q3G Mixed Micelles:

It has also been observed that both hyperoside and Q3G are beta-adrenergic antagonists. This quality is relevant because there have been at least six retrospective studies that have consistently demonstrated a connection between beta-blocker use (and beta-2 antagonist propranolol, in particular) and about a 50% reduction in the occurrence of metastatic breast cancer (and triple negative breast cancer (TNBC), in particular) (See, e.g., a) Melhem-Bertrandt, “Beta-blocker use is associated with improved relapse-free survival in patients with triple-negative breast cancer,” J Clin Oncol, 29: 2645-2652, 2011; b) Barron, “Beta blockers and breast cancer mortality: A population-based study,” J Clin Oncol, 29: 2635-2644, 2011; c) Powe, “Beta-blocker drug therapy reduces secondary cancer formation in breast cancer and improves cancer specific survival,”. Oncotarget, 1: 628-638, 2010; d) Botteri, “Therapeutic effect of β-blockers in triple-negative breast cancer postmenopausal women,” Breast Cancer Res. Treat. 2013 August; 140 (3):567-75, and e) Choy, “Inhibition of β2-adrenergic receptor reduces triple-negative breast cancer brain metastases: The potential benefit of perioperative β-blockade,” Oncology Reports, 35, 2016, 3135-42; and f) Parada-Huerta, “Metastasis Risk Reduction Related with Beta-Blocker Treatment in Mexican Women with Breast Cancer,” Asian Pac. J. Cancer Prev., 2016; 17(6):2953-7).

In addition, beta-blockers also appear to increase the survival of ovarian cancer patients (Sood, “Clinical impact of selective and nonselective beta-blockers on survival in patients with ovarian cancer,” Cancer, 121, 2015). Currently, an interventional study is being conducted at the MD Anderson Cancer Center to examine the effect of a nonselective β-blocker plus standard chemotherapy (paclitaxel and carboplatin or possibly docetaxel) to treat ovarian cancer. “Feasability Study: Therapeutic Targeting of Stress Factors in Ovarian cancer Patients”. NCT01504126

It has been reported that quercetin-3-O-glucuronide (Q3G), a metabolite of the phytochemical quercetin, is also a beta-adrenergic antagonist (Yamazaki, Arch. Biochem. Biophys. 2014 Sep. 1; 557:18-27). Yamazaki reported that Q3G (0.1 μM) suppressed invasion of MDA-MB-231 breast cancer cells (which are TNBC cells) and MMP-9 induction, and inhibited the binding of [(3)H]-NA to β2-AR. Yamazaki concluded that Q3G may function to suppress invasion of breast cancer cells by controlling β2-adrenergic signaling, and may be a dietary chemopreventive factor for stress-related breast cancer. Based upon its behavior as a beta-adrenergic antagonist and its exemplary safety profile that is essentially free of side effects, it is proposed herein to use Q3G and/or hyperoside in the self-assemblies described above as a chemotherapeutic for TNBC and ovarian cancers in patients already diagnosed with these cancers in order to prevent metastatic breast or ovarian cancer.

b. Propranolol Liposomes and MLVs:

It has also been further been observed the positive results in the above-mentioned cancer epidemiology studies concerning beta-blockers appear to correlate strongly with the specific use of propranolol. Although propranolol has been successfully used to treat hypertension and is on the WHO List of Essential Medicines, it nonetheless has some drawbacks in that its relatively rapid metabolism often requires that it be taken 2-4 times daily and its extent of metabolism is inconsistent across different patients, thereby requiring a lengthy titration procedure at the beginning of the therapy. Therefore, it is another goal to improve the metabolic and pharmacokinetic profile of propranolol.

To this end, it is observed that the literature has reported that propranolol is sufficiently amphiphilic as to form micelles, with a critical micelle concentration (CMC) of about 0.13 mol/L (Ruso, J. Chem. Eng. Data, 2003, 48(6), pp 1597-1602). See also Schreier, Biochimica et Biophysica Acta, 1508 (2000) 210-234 for reports of propranolol micelles. This amphiphilic quality of propranolol makes it reasonable to expect that the skilled artisan can also make self-assembled propranolol structures including gels, multi-lamellar vesicles (MLVs) and liposomes. These complex self-assembled propranolol structures can then be used to treat TNBC and ovarian cancer patients and thereby prevent metastatic cancer.

c. Ginsenoside Liposomes and MLVs:

It has further been reported that ginsenosides appear to be efficacious in treating cancers and especially lung cancer. In particular, one set of investigators has reported that administration of one particular ginsensoide (Rg3) to lung cancer patients has significantly increased the postoperative life span of those patents, and that Rg3 performed substantially as well as standard chemotherapy (Lu, Chin. J. Integr. Med., 2008 Mar. 14(1) 33-6). Lu further reported special efficacy of Rg3 against patients having a “positive VEGF expression” phenotype. Another set of investigators has reported that the combination of the ginsenoside Rg3 along with EGFR-TKI chemotherapy produced a 20% increase in the duration of progression free survival in lung cancer patients (Li, Oncotarget, 2016 Sep. 16). Therefore, in some embodiments, the complex self-assembled ginsenosides self-assemblies discussed above are administered to lung cancer patients, preferably through the pulmonary route.

d. Baicalin and COPD:

Baicalin has also been demonstrated to be an inhibitor of prolyloligopeptidase (POP) (Tarrago, Bioorg. Med. Chem., 2008 Aug. 1; 16(15):7516-24). Tarrago reported that baicalin inhibited prolyloligopeptidase in a dose-dependent manner, with inhibition experiments using baicalin analogs showing that the sugar moiety was not necessary for activity. The IC₅₀ of baicalin and its aglycone derivative baicalein were rather similar, showing that the sugar moiety was not involved in the interaction of baicalin with POP.

This anti-POP feature of baicalin may signal a utility of baicalin in preventing lung cancer, as it has been reported that cigarette smoke-induced lung emphysema in mice is associated with POP, an enzyme associated with collagen breakdown (Braber, Am J Physiol Lung Cell Mol Physiol. 2011 February; 300(2):L255-65). One clinical trial in which a POP inhibitor (Roflumilast) was provided to COPD patients reported reduced pulmonary inflammation through decreasing prolyl endopeptidase activity and AcPGP. The investigators correlated lower AcPGP levels with blunted markers of neutrophilic inflammation, and concluded that inhibiting this self-propagating pathway lessens the overall inflammatory burden, which may alter the natural history of COPD, including the risk of exacerbation (Wells, Am. J. Respir. Crit. Care Med., 2015 Oct. 15; 192(8):934-42) (NCT 01572948).

Therefore, there is provided a method of treating a patient with COPD (preferably a smoker with COPD), comprising the steps of (a) providing an inhaler housing a formulation comprising spray-dried baicalin nanoparticles and a mucoadhesive excipient, and (b) carrying out a pulmonary administration of the formulation to the patient.

Ganglioside-Containing Vesicles

In some embodiments, the self-assembled structures can be coated with a layer of a mucoadhesive (such as pectin) in order to enhance the binding of the self-assembly to the wall of the GI tract or lung.

Liposomes are often used to orally deliver drugs to the circulation (Ahmad, Curr Drug Metab., 2015; 16(8): 633-44). Oral lipsomes are typically made of amphiphilic lecithin, which contains a hydrophilic head group and hydrophobic tails. Lecithins are usually phospholipids, composed of phosphoric acid with choline, glycerol or fatty acids, usually glycolipids or triglyceride. Glycerophospholipids in lecithin include phosphatidylcholine, phosphatidyletahanolamine, phosphatidylinositol, phosphatidylserine and phosphatidic acid.

Although lecithin-based liposomes have been routinely used to orally deliver drugs, their use presents three challenges. First, lecithin-based liposomes do not robustly survive the acidity and bile present in the gastrointestinal tract (Taira, Drug Delivery, 11, 2; 123-128 (2004)). Second, lecithin-based liposomes typically release their contents so quickly as to require multiple dosings per day for molecules with short half-lives. Third, lecithin-based liposomes that enter circulation are often susceptible to quick removal by RES uptake (Litzinger, Biochim. Biophys. Acta., 1992 Feb. 17, 1104(1)179-87).

Gangliosides are endogenous amphipathic molecules, and are present in human milk. Recently, gangliosides have been found to be highly important molecules in immunology. Natural and semisynthetic gangliosides are considered possible therapeutics for neurodegenerative disorders. See, for example, Mocchetti I (2005). “Exogenous gangliosides, neuronal plasticity and repair, and the neurotrophins,” Cell Mol Life Sci. 62(19-20):2283-94. Accordingly, orally-delivered gangliosides should be considered safe and even beneficial for mother and infant. Indeed, gangliosides have even been provided to 2230 children suffering from cerebral palsy, with the reported result of improved neurological symptoms (Xu, Chin. J. Clin. Rehab., 2005, 9, 122-123).

It has been recently found that adding gangliosides to the conventional lecithin-like liposome helps delivery of that active to the patient. First, they increase the robustness of the liposome towards the GI tract: “This study suggests that among the formulations used as oral drug carriers, those containing GM1 and GM type III have higher possibilities of surviving through the gastrointestinal tract” (Taira, Drug Delivery, 11, 2; 123-128 (2004)). Second, ganglioside-containing lecithin liposomes that enter circulation are less susceptible to quick removal by RES uptake, thereby prolonging their lifetime in circulation (Chonn, J. Liposome Research, 2(3), 397-410, 1992). Third, adding gangliosides to the conventional liposomes can reduce the flux of glucose-6-phosphate (G6P) from the liposome into plasma to a level of about 5% per hour (see FIG. 2 of Taira), thereby allowing for nearly constant release of G6P from the liposome over the course of one day. Therefore, it is believed that gangliosides beneficially reduce the gaps in the liposome structure to reduce the flux of low molecular weight, hydrophilic molecules like G6P therethrough.

G6P has a molecular weight of about 260 daltons and a log P of −3.24. It is believed that other low MW molecules that are likewise hydrophilic should pass through a ganglioside-containing lecithin-based liposome with a comparable flux, thereby allowing for once a day dosing and a constant plasma concentration.

In particular, it is believed that molecules having a molecular weight of between about 100 and 400 daltons) and that are likewise hydrophilic (preferably a log P<0), should pass through a ganglioside-containing lecithin-based liposome with a comparable flux as G6P, thereby allowing for once a day dosing and a constant plasma concentration.

Preferably, it is believed that molecules having a molecular weight of between about 200 and 400 daltons, and between 200 and 300 daltons in some embodiments, and between 225 and 275 daltons in others) and that are likewise very hydrophilic (log P<−1), should pass through a ganglioside-containing lecithin-based liposome with a comparable flux as G6P, thereby allowing for once a day dosing and a constant plasma concentration.

More preferably, it is believed that molecules having a molecular weight of between about 100 and 400 daltons), have a cyclic component and are very hydrophilic (log P<0), should pass through a ganglioside-containing lecithin-based liposome with a comparable flux as G6P, thereby allowing for once a day dosing and a constant plasma concentration.

Taira's embodiments that displayed the desired flux used lecithin, cholesterol, sphingomyelin and ganglioside in a 1:1:1:0.14 molar ratio. Therefore, in some embodiments, the ganglioside-containing lecithin-based liposome comprises between 10 mol % and 50 mol % lecithin. In some embodiments, the ganglioside-containing lecithin-based liposome comprises between 1 mol % and 10 mol % ganglioside. In some embodiments, the ganglioside-containing lecithin-based liposome comprises between 10 and 50 mol % lecithin and between 1 mol % and 10 mol % ganglioside.

In some embodiments, the ganglioside-containing lecithin-based liposome comprises (a) between 10 mol % and 50 mol % lecithin (preferably between 20 mol % and 30 mol %), (b) between 10 mol % and 50 mol % cholesterol (preferably between 20 mol % and 30 mol %), (c) between 10 mol and 50 mol % sphingomyelin (preferably between 20 mol % and 30 mol %), (d) between 1 mol % and 10 mol % ganglioside, and (e) an anti-depressant.

Preferably, the anti-depressant is characterized by (a) a molecular weight of between about 100 and 400 daltons, preferably between 200 and 400 daltons, more preferably between 200 and 300 daltons, (b) hydrophilicity (preferably a log P<0, more preferably a log P<−1), and (c) (optionally) a cyclic component.

In some embodiments, the ganglioside is selected from the group consisting of GM1 and GM type III. These were the gangliosides used by Taira to obtain good GI robustness and optimal G6P flux in plasma. GM1 is found in mother's milk in a concentration of between 0.02% and 0.77% of the total lipid-bound sialic acid. GM1 produces antidepressant effects in mice through a BDNF signaling cascade (Jiang, Int. J. Neuropsychopharmacology, 2016, 19(9) 1-13). It is believed that GM type III is a mixture of 20% sialic acid, and equimolar amounts of GM1 and GDla gangliosides.

Thyroid-Releasing Hormone(TRH)

In one embodiment, the active agent is TRH. TRH is available as a supplement (Abaris). It is a clinically demonstrated as a lactation enhancer (U.S. Pat. No. 4,125,605; United States as assignee), and so should be safe for the breastfeeding infant. A rapid antidepressant response after nocturnal TRH administration has been demonstrated in patients with bipolar type I and bipolar type II major depression (Szuba, J Clin Psychopharmacol. 2005 August; 25(4):325-30). Therefore, TRH should be beneficial for the perinatally depressed mother. TRH has a molecular weight of about 362 daltons, is very hydrophilic (log P=−2.46), and it has a short half-life (7.6 minutes). Duntas, Thyroidology. 1991 May; 3(2):51-7. See also Bassiri, J. Clin. Invest., 52, July 1973, 1616-19. Therefore, its simple once-a-day oral delivery does not dispose itself to a relatively constant plasma concentration over the course of a day.

Therefore, TRH should pass through a ganglioside-containing lecithin-based liposome into plasma with a flux comparable to G6P, thereby allowing for once a day dosing and a constant plasma concentration.

Cyclo His-Pro

In another embodiment, the active agent suitable for once-a-day dosing through a ganglioside-containing lecithin-based liposome is Cyclo His-Pro. Cyclo His-Pro is a major TRH metabolite. It has been called “an important new tool in counteracting neuroinflammation-based degenerative pathologies” Grotelli, Intl. J. Molec. Sci., 2016, 17, 1332. Accordingly, it should be beneficial to a perinatally depressed mother whose condition is characterized by an inflammation related phenotype. It can also be useful in treating gestational diabetes, as it (with histidine) decreases blood glucose concentrations in type 2 diabetic mice (Hwang, Diabetes Obes. Metab., 2003 September, 5(5), 317-24), and protects pancreas cells from apoptosis (Koo, J. Microbiol. Biotechnol., 2011 February, 21(2) 218-27). Cyclo His-Pro has a molecular weight of about 234 daltons, is very hydrophilic (Log P=−1.48) and has a short biphasic half-life of 1 and 33 minutes (Koch, Biochem. Biophys. Res. Comm., 104(2), 1982, 823-9). Therefore, simple once-a-day oral delivery does not dispose itself to a relatively constant plasma concentration over the course of a day.

Therefore, Cyclo His-Pro should pass through a ganglioside-containing lecithin-based liposome into plasma with a flux comparable to G6P, thereby allowing for once a day dosing and a constant plasma concentration.

Taurine

Taurine is the major ingredient in popular energy drinks, and is added to many infant formulas (wikipedia). Taurine is an essential amino acid for pre-term neonates (Lourenco, Nutr. Hosp., 2002, XVII, 6, 262-270). It is very hydrophilic (Log P=−3.36) and its oral administration in healthy volunteers is characterized by a short half-life of 1 hour (Ghandforoush-Sattari, J. Amino Acids, Vol. 2010, 346237). Therefore, simple once-a-day oral delivery does not dispose itself to a relatively constant plasma concentration over the course of a day. Lastly, the antidepressant effect of chronic taurine administration has been demonstrated in rats (Toyoda, Adv. Exp. Med. Biol., 2013, 775, 29-43). Accordingly, it should be beneficial to a perinatally depressed mother.

Therefore, Taurine should pass through a ganglioside-containing lecithin-based liposome into plasma with a flux comparable to G6P, thereby allowing for once a day dosing and a constant plasma concentration.

Pyroglutamyl Leucine (PGL)

PGL is a wheat-hydrolysate, and so should be safe for an infant. PGL provides an antidepressant effect in mice through enhancing hippocampal neurogenesis (Yamamoto, Neuropeptides, 2015 June, 51, 25-9), and so can be used for a perinatally-depressed mother. PGL is also anti-inflammatory (Hirai, Life Sci., 2014 Nov. 4, 117(1) 1-6), and so should be of particular use for a perinatally-depressed mother having an inflammatory phenotype. The high number of nitrogen and COOH groups in the small molecule makes it reasonable to conclude that PGL is very hydrophilic. The fact that it is a peptide subject to rapid amidase activity makes it reasonable to conclude that PGL has a short half-life. Therefore, simple once-a-day oral delivery does not dispose itself to a relatively constant plasma concentration over the course of a day.

Therefore, PGL should pass through a ganglioside-containing lecithin-based liposome into plasma with a flux comparable to G6P, thereby allowing for once a day dosing and a constant plasma concentration.

Carnosine

Carnosine is available as a supplement, and is highly concentrated in brain. Carnosine is very hydrophilic, with a log p=−1.19. It has a short half-life in human plasma<5 minutes (Gardner, J. Physiology, 1991, 439, 411-422). Therefore, simple once-a-day oral delivery does not dispose itself to a relatively constant plasma concentration over the course of a day. Carnosine is credited for the antidepressant effect of chicken breast extract in rats (Tomonaga, Pharmacol Biochem Behav., June 2008, 89, 4, 627-32). Accordingly, it should be beneficial to a perinatally depressed mother. Carnosine also significantly improved symptoms of autism in children (Chez, J. Child Neurol., 2002 November, 17(11), 833-7).

Because of its relatively low molecular weight and high hydrophilicity, carnosine should pass through a ganglioside-containing lecithin-based liposome into plasma with a flux comparable to G6P, thereby allowing for once a day dosing and a constant plasma concentration.

Alanyl-Glutamine (AG)

AG is available as a supplement (Sustamine™). AG protects against ischemia-reperfusion injury by upregulating bcl-2 (Jia, World J. Gastroenterol., 2006 Mar. 7, 12(9), 1373-8). Its ability to increase bcl-2 makes it reasonable to conclude that AG will help increase neuronal synaptic plasticity, and so makes AG an attractive candidate for antidepression therapy. AG has a plasma half-life in ICU patients of 0.26 hours (Berg, Amino Acids. 2005 November; 29(3):221-8), and so does not remain in blood very long. Therefore, simple once-a-day oral delivery does not dispose itself to a relatively constant plasma concentration over the course of a day. This leads one to conclude that it requires multiple dosings per day. AG has a log P=−2.15, and so is very lipophilic. AG has been used in double-blind trials in infants (Struijs, Clin Nutr. 2013 June; 32(3):331-7), and so is likely safe for infants. The three nitrogen atoms in the molecules leads one to reasonably conclude that it is very hydrophilic.

Because of its relatively low molecular weight and high hydrophilicity, AG should pass through a ganglioside-containing lecithin-based liposome into plasma with a flux comparable to G6P, thereby allowing for once a day dosing and a constant plasma concentration.

Glutaurine(Gamma-Glutamyltaurine) (GGT)

GGT is very hydrophilic, with a Log P=−3.36. GGT is a potent anti-epileptic in amygdala-kindled rats (Uemura, Brain Res., 1992 October, 594(2), 347-50), and it modulates excitatory neurotransmission in vitro (Varga, Neurochem. Res., 1994 March, 19(3), 243-8). Therefore, GGT is an attractive candidate for a mother who suffers from epilepsy. GGT is available as a supplement in Hungary (Litoralon). GGT is thought to affect emotional arousal and is considered to be an anti-conflict molecule (Bittner, Amino Acids, 2005 June, 28(4), 343-56).

Because of its relatively low molecular weight and high hydrophilicity, A-G should pass through a ganglioside-containing lecithin-based liposome into plasma with a flux comparable to G6P, thereby allowing for once a day dosing and a constant plasma concentration.

Non-PND Molecules

Cytarabine:

Cytarabine is mainly used in the treatment of leukemia and lymphomas, where it is the backbone of induction chemotherapy. It is on the World Health Organization's List of Essential Medicines. The half-life of subcutaneously delivered cytarabine is 18 minutes, while its half-life in plasma via a continuous infusion is 2.1 hours (Liliemark, Semin. Oncol., 1987 June, 14 (2 supp 1) 167-71). Harris reports that its intravenously delivered half-life is 7-107 minutes (Harris. Br. J. Pharmacol., 1979 September, 8(3) 219-27). Therefore, its simple once-a-day oral delivery does not dispose itself to a relatively constant plasma concentration over the course of a day. Cytarabine is very hydrophilic, with a log P=−2.8.

Because of its relatively low molecular weight, cyclic structure and high hydrophilicity, cytarabine should pass through a ganglioside-containing lecithin-based liposome into plasma with a flux comparable to G6P, thereby allowing for once a day dosing and a relatively constant plasma concentration.

Cytarabine is a cytidine. According to Wikipedia, a cytidine is a nucleoside molecule that is formed when cytosine is attached to a ribose ring via a β-N₁-glycosidic bond. Cytidine is a component of RNA. If cytosine is attached to a deoxyribose ring, it is known as a deoxycytidine. Therefore, in some embodiments, there is provided a ganglioside-containing lecithin-based liposome comprising (a) between 10 mol % and 50 mol % lecithin (preferably between 20 mol % and 30 mol %), (b) between 10 mol % and 50 mol % cholesterol (preferably between 20 mol % and 30 mol %), (c) between 10 mol and 50 mol % sphingomyelin (preferably between 20 mol % and 30 mol %), and (d) between 1 mol % and 10 mol % ganglioside, and (e) a cytarabine.

Compositions and Methods of Treatment

The invention further relates to therapeutically effective amounts of the composition for use in treating a subject with a GABA(A) delta agonist, preferably the subject suffers from perinatal depression. The methods comprise administering to the subject a therapeutically effective amount of compositions according to embodiments of the invention. The compositions preferably further comprise a pharmaceutically acceptable carrier. In the present context, the term “pharmaceutically acceptable” means that the carrier, at the dosages and concentrations employed, will not cause unwanted or harmful effects in the subjects to which they are administered. Such pharmaceutically acceptable carriers and excipients are well known in the art (see Remington's Pharmaceutical Sciences, 18^{th} edition, A. R. Gennaro, Ed., Mack Publishing Company [1990]; Pharmaceutical Formulation Development of Peptides and Proteins, S. Frokjaer and L. Hovgaard, Eds., Taylor & Francis [2000]; and Handbook of Pharmaceutical Excipients, 3^{rd} edition, A. Kibbe, Ed., Pharmaceutical Press [2000]). The term “carrier” refers to a diluent, adjuvant, excipient, or vehicle with which the composition is administered. Saline solutions and aqueous dextrose and glycerol solutions can, e.g., be employed as liquid carriers, particularly for injectable solutions. The exact formulation should suit the mode of administration. The compositions are preferably formulated and administered as a sterile solution. Sterile solutions are prepared by sterile filtration or by other methods known per se in the art. The solutions can then be lyophilized or filled into pharmaceutical dosage containers. The pH of the solution generally is in the range of pH 3.0 to 9.5, e.g., pH 5.0 to 7.5.

Administration of the compositions according to the invention can be performed using standard routes of administration. Non-limiting examples include oral administration; pulmonary administration; parenteral administration, such as intravenous, intradermal, transdermal, intramuscular, or subcutaneous; inhalation; and mucosal administration, e.g., intranasal, vaginal, rectal, or sublingual routes of administration. The compositions can be formulated for each route of administration, see, e.g., International Publication Nos. WO 1993/25221 (Berstein et al.) and WO1994/17784 (Pitt et al.), the relevant content of which is incorporated herein by reference.

Solid dosage forms for oral administration include capsules, tablets, pills, powders, and granules. In such solid dosage forms, the active compound is admixed with at least one pharmaceutically acceptable carrier such as sucrose, lactose, or starch. Such dosage forms can also comprise, as is normal practice, additional substances other than inert diluents, e.g., lubricating agents such as magnesium stearate. In the case of capsules, tablets, and pills, the dosage forms can also comprise buffering agents. Tablets and pills can additionally be prepared with enteric coatings.

Liquid dosage forms for oral administration include pharmaceutically acceptable emulsions, solutions, suspensions, syrups, with the elixirs containing inert diluents commonly used in the art, such as water. Besides such inert diluents, compositions can also include other agents, such as wetting agents, emulsifying and suspending agents, and sweetening, flavoring, and perfuming agents.

Preparations for parental administration include sterile aqueous or non-aqueous solutions, suspensions, or emulsions. Examples of non-aqueous solvents or vehicles are propylene glycol, polyethylene glycol, vegetable oils, such as olive oil and corn oil, gelatin, and injectable organic esters such as ethyl oleate. Such dosage forms can also contain other agents such as preserving, wetting, emulsifying, and dispersing agents. They can be sterilized by, for example, filtration through bacteria retaining filter, by incorporating sterilizing agents into the compositions, by irradiating the compositions, or by heating the compositions. They can also be manufactured using sterile water, or some other sterile injectable medium immediately before use.

Administration of the compositions disclosed herein are typically topical, oral, mucosal, or by inhalation. Typically, administration will have a therapeutic and/or prophylactic aim to treat or prevent perinatal depression in the subject in need thereof. In therapeutic applications, the compositions are administered to a subject already dealing with perinatal depression in an amount sufficient to cure or at least partially provide a reduction in the symptoms of the perinatal depression. In prophylactic applications, the compositions are administered to the subject that is susceptible to-or at risk of perinatal depression (i.e., during the later stages of pregnancy or right after the subject gives birth). In each of these scenarios the amount of the composition will depend on the state of the subject (e.g., severity of the symptoms of perinatal depression) and the physical characteristics of the subject (e.g., height, weight, etc.).

The actual amount administered, and rate and time-course of administration, will depend on the nature and severity of what is being treated. Prescription of treatment, e.g., decisions on dosage etc., is within the responsibility of general practitioners and other medical doctors, or in a veterinary context a veterinarian, and typically takes account of the disorder to be treated, the condition of the individual patient, the site of delivery, the method of administration and other factors known to practitioners. Examples of the techniques and protocols mentioned above can be found in Remington's Pharmaceutical Sciences, 16th edition, Osol, A. ed., 1980.

### EMBODIMENTS

The invention provides also the following non-limiting embodiments.

Embodiment 1 is a composition comprising (a) a neurosteroid; and (b) a saponin in an amount effective to form a self-assembled structure incorporating the neurosteroid.

Embodiment 2 is the composition of embodiment 1, wherein the self-assembled structure is a micelle, a gel, a liposome, a lamellar phase, or a multi-lamellar vesicle.

Embodiment 3 is the composition of embodiment 1 or 2, wherein the self-assembled structure is a micelle.

Embodiment 4 is the composition of any one of embodiments 1-3, wherein the saponin is selected from the group consisting of a soyasaponin, a quillaja saponin, and a ginsenoside saponin.

Embodiment 5 is the composition of any one of embodiments 1-4, wherein the neurosteroid is selected from the group consisting of an allopregnanolone, a tetrahydrodeoxycorticosterone (THDOC), and a progesterone.

Embodiment 6 is the composition of embodiment 5, wherein the neurosteroid is an allopregnanolone.

Embodiment 7 is the composition of any one of embodiments 1-6, wherein the saponin surfactant is at least about 0.1 wt %, at least about 0.5 wt %, or at least about 1 wt % relative to the total weight of the composition.

Embodiment 8 is the composition of any one of embodiments 1-7, wherein the composition comprises at least about 100 parts per million, at least about 200 parts per million, or at least about 300 parts per million of the neurosteroid.

Embodiment 9 is the composition of any one of embodiments 1-8, wherein the composition further comprises a pharmaceutically acceptable carrier.

Embodiment 10 is the composition of any one of embodiments 1-9, wherein the composition further comprises an adjuvant.

Embodiment 11 is the composition of any one of embodiments 1-10, wherein the composition has a permeation coefficient P of about 0.01 per hour to about 0.05 per hour.

Embodiment 12 is the composition of embodiment 11, wherein the composition has a permeation coefficient P of about 0.01 per hour.

Embodiment 13 is a method of treating a subject, preferably a subject in need of a treatment of a perinatal depression (PND), the method comprising administering to the subject a therapeutically effective amount of the composition of any one of embodiments 1-12.

Embodiment 14 is the method of embodiment 13, wherein the therapeutically effective amount of the composition is administered topically, intravenously, orally, mucosally, or by inhalation.

Embodiment 15 is the method of embodiment 13 or 14, wherein the therapeutically effective amount of the composition is administered in a single dose, once every 4 hours, preferably once every 8 hours, more preferably once every 12 hours, most preferably once every 24 hours or a longer period of time.

Embodiment 16 is the method of any one of embodiments 13-15, wherein the therapeutically effective amount of the composition has a permeation coefficient P of about 0.01 per hour to about 0.05 per hour.

Embodiment 17 is the method of embodiment 16, wherein the therapeutically effective amount of the composition has a permeation coefficient P of about 0.01 per hour.

Embodiment 18 is the method of any one of embodiments 13-17, wherein the therapeutically effective amount of the composition has a permeation coefficient P at least two times, at least five times, or at least ten times lower than a permeation coefficient P of a composition comprising a monomolecular neurosteroid or a cyclodextrin neurosteroid complex.

Embodiment 19 is a method of producing the composition of any one of embodiments 1-12, the method comprising adding a solution of the neurosteroid dissolved in an organic solvent to a saponin solution under flow, wherein the neurosteroid is incorporated into the self-assembled structure.

Embodiment 20 is the method of embodiment 19, wherein the organic solvent dissolves at least 0.1 wt %, at least 0.5 wt %, or at least 1 wt % of the neurosteroid.

Embodiment 21 is the method of embodiment 19 or 20, wherein at least 1 wt %, at least 2.5 wt %, or at least 3 wt % of the organic solvent is soluble in water.

Embodiment 22 is the method of an one of embodiments 19-21, wherein the organic solvent is selected from the group consisting of ethanol, methanol, propanol, butanol, glycol, ethylene glycol, propylene glycol, butylene glycol, diethyl ether, and mixtures thereof.

Embodiment 23 is the method of embodiment 22, wherein the organic solvent is ethanol.

Embodiment 24 is a method of making a concentrated hydroxyfatty acid fraction from a DHA-containing fluid comprising i) low melting point fatty acids and ii) high melting point fatty acids, wherein both acids are present in triglycerides, comprising the steps of:

a) freezing the fluid to separate out the low melting point fatty acids from the high melting point fatty acids contained therein and thereby produce a low melting point-enriched fluid comprising parent triglycerides,

b) ex vivo contacting the low melting point-enriched fluid with PLA2 to selectively free hydroxyfatty acids from their triglycerides to produce a free hydroxyfatty acid-enriched fluid, and

c) ex vivo contacting the free hydroxyfatty acid-enriched fluid with a concentrator to selectively capture the freed hydroxyfatty acids within the concentrator to produce a concentrator having hydroxyfatty acid adsorbed thereon.

Embodiment 25 is the method of embodiment 24, wherein the freezing step is carried out at about −20° C. to −40° C.

Embodiment 26 is the method of embodiment 24, wherein the fluid is a marine oil.

Embodiment 27 is the method of embodiment 24, wherein the fluid is an algae oil.

Embodiment 28 is the method of embodiment 24, wherein the fluid is derived from a milk.

Embodiment 29 is the method of embodiment 28, wherein a step of separating out a fat fraction of the milk is carried out prior to step a), and step b) is carried out on the separated fat fraction.

Embodiment 30 is the method of embodiment 24, wherein the concentrator is present as a cyclodextrin carbonate nanoparticle.

Embodiment 31 is the method of embodiment 24, wherein the concentrator is selected from the group consisting of cyclodextrin, zeolite, mesoporous silica, and octadecyl silyl silica (OSS).

Embodiment 32 is the method of embodiment 24, wherein the concentrator is cyclodextrin,

Embodiment 33 is the method of embodiment 24, wherein the concentrator is zeolite.

Embodiment 34 is the method of embodiment 24, wherein the concentrator is mesoporous silica.

Embodiment 35 is the method of embodiment 24, wherein the concentrator is octadecyl silyl silica (OSS).

Embodiment 36 is the method of embodiment 24 further comprising the step of:

d) administering the concentrator having hydroxyfatty acid adsorbed thereon to a human.

Embodiment 37 is the method of embodiment 36, wherein the concentrator having hydroxyfatty acid adsorbed thereon is enriched in adsorbed 17-OH DHA.

Embodiment 38 is the method of embodiment 24, wherein the concentrator is a solid porous body, and the hydroxyfatty acid is adsorbed within the porosity of the concentrator.

Embodiment 39 is the method of embodiment 24, wherein the PLA2 is immobilized PLA2.

Embodiment 40 is the method of embodiment 24, further comprising the steps of:

d) releasing the hydroxyfatty acid from the concentrator to produce a hydroxyfatty acid-enriched solution, and

e) administering the hydroxyfatty acid-enriched solution to a human.

Embodiment 41 is a method of making a concentrated hydroxyfatty acid fraction from a DHA-containing fluid comprising triglycerides comprising hydroxyfatty acids, comprising the steps of:

a) ex vivo contacting the fluid with PLA2 to selectively free hydroxyfatty acids from their triglycerides and thereby produce a free hydroxyfatty acid-enriched fluid, and

b) ex vivo contacting the free hydroxyfatty acid-enriched fluid with a concentrator to selectively capture the freed hydroxyfatty acids within the concentrator to produce a concentrator having hydroxyfatty acid adsorbed thereon.

Embodiment 42 is the method of embodiment 41, further comprising the step of:

c) administering the concentrator having hydroxyfatty acid adsorbed thereon to a human.

Embodiment 43 is the method of embodiment 41, further comprising the steps of:

c) releasing the hydroxyfatty acid from the concentrator to produce a hydroxyfatty acid-enriched solution, and

d) administering the hydroxyfatty acid-enriched solution to a human.

Embodiment 44 is the method of embodiment 41, wherein the PLA2 is immobilized.

Embodiment 45 is the method of embodiment 41, wherein the concentrator having hydroxyfatty acid adsorbed thereon is selectively removed from the free hydroxyfatty acid-enriched fluid.

Embodiment 46 is the method of embodiment 41, wherein the concentrator is selected from the group consisting of cyclodextrin, zeolite, mesoporous silica, and octadecyl silyl silica (OSS).

Embodiment 47 is the method of embodiment 41, further comprising:

c) separating the concentrator having hydroxyfatty acid adsorbed thereon from the fluid.

Embodiment 48 is the method of embodiment 47, further comprising the step of:

d) administering the separated concentrator having hydroxyfatty acid adsorbed thereon to a human.

Embodiment 49 is the method of embodiment 48, wherein the administration is carried out once a day.

Embodiment 50 is a method of making a concentrated hydroxyfatty acid fraction from a DHA-containing fluid comprising esters comprising hydroxyfatty acids, comprising the steps of:

a) ex vivo contacting the fluid with an enzyme to selectively free hydroxyfatty acids from their esters and thereby produce a free hydroxyfatty acid-enriched fluid, and

b) ex vivo contacting the free hydroxyfatty acid-enriched fluid with a concentrator to selectively capture the freed hydroxyfatty acids within the concentrator to produce a concentrator having hydroxyfatty acid adsorbed thereon.

### EXAMPLES

### Preparation of Inventive Examples and Comparative Examples

Inventive Examples and Comparative Examples were prepared utilizing different types of formulation ingredients (i.e. raw materials from various suppliers). These materials, along with INCI/material names, CAS/item number, and suppliers are listed below:

Allopregnanolone (AP):

AP CS was obtained as 3β-hydroxy-5α-pregnan-20-one Allopregnanolone, CAS#516-55-2, from CarboSynth LLC (CS); Berkshire, UK.

AP ST was obtained as 3α-hydroxy-5α-pregnan-20-one Allopregnanolone, CAS#516-54-1, from Steraloids Inc. (ST); Newport, R.I., USA.

Saponins:

VaxSap saponin was obtained as Vax-Sap (Saponin), purified quillaja saponaria, item#20-2-100-005, from Desert King International; San Diego, Calif., USA.

Soyasaponin I, CAS#51330-27-9, item# S9951, and Ginsenoside Rb1, CAS#41753-43-9, item#00170580, and Ginsenoside Rg1, CAS#22427-39-0, item#1291672 were obtained from Sigma-Aldrich Co. LLC; St. Louis, Mo., USA.

Other:

H-b-Cyclodextrin was obtained as (2-hydroxypropyl)-beta-cyclodextrin, CAS#128446-35-5, item# C0926, Mw=1396 g/mol, from Sigma-Aldrich Co. LLC.

Castor oil, CAS#8001-79-4, item#18722, was obtained from Sigma-Aldrich Co. LLC.

Ethanol was obtained as Pure 190 Ethanol, USP Excipient, item#017635, from Archer Daniels Midland Company; Chicago, Ill., USA.

Propylene glycol, USP, CAS#57-55-6, item#1576708, was obtained from Sigma-Aldrich Co. LLC.

Deionized water (DI water) was obtained from a Millipore Direct-Q™ System with Progard™ 2 filter.

Composition and Method of Making

Convection-driven Solvent-to-Water Complexation (CSWC) Method: Water-insoluble allopregnanolone was discovered to be capable of incorporation into saponin surfactant micelles by adding an allopregnanolone solvent solution to an aqueous solution of saponin surfactant under flow. The solvent solution comprised ethanol; however additional organic solvents such as, but not limited to, methanol, propanol, butanol, ethylene glycol, propylene glycol, diethyl ether, or mixtures thereof could be used for the solvent solution, such that an adequate amount of allopregnanolone was dissolved. The saponin surfactant concentration in water was above the critical micelle concentration (cmc). The allopregnanolone solvent solution was added with the saponin surfactant solution and mixed quickly such that the diffusion driven incorporation of the allopregnanolone into the saponin micelles was favored over the crystallization of allopregnanolone.

### Preparation of Inventive Examples E1-E7 and Comparative Examples C1-C7

Inventive Examples E1-E7 were prepared as follows: To 10 g solution of surfactant in DI water placed in a 20 mL scintillation vial the appropriate amount of an allopregnanolone solution in ethanol was added dropwise under stirring with a magnetic stirrer at 200 rpm at 22+/−1 degree Celsius. The vial was covered after the addition and the solution was stirred for 30 minutes.

Comparative Examples C1 and C2 were prepared as follows: To 10 g solution of DI water or surfactant in DI water placed in a 20 mL scintillation vial the appropriate amount of allopregnanolone crystals were added under stirring with a magnetic stirrer at 200 rpm at 22+/−1 degree Celsius. The vial was covered after the addition and the solution was stirred for 24 hours.

Comparative Examples C3 and C4 were prepared as follows: To 10 g solution of DI water placed in a 20 mL scintillation vial the appropriate amount of an allopregnanolone solution in ethanol was added dropwise under stirring with a magnetic stirrer at 200 rpm at 22+/−1 degree Celsius. The vial was covered after the addition and the solution was stirred for 30 minutes.

Comparative Examples C5 and C6 were prepared as follows: To 10 g solution of H-b-Cyclodextrin in DI water placed in a 20 mL scintillation vial the appropriate amount of an allopregnanolone solution in ethanol was added dropwise under stirring with a magnetic stirrer at 200 rpm at 22+/−1 degree Celsius. The vial was covered after the addition and the solution was stirred for 30 minutes.

Comparative Example C7 was prepared as follows: To 10 g of propylene glycol placed in a 20 mL scintillation vial the appropriate amount of an allopregnanolone solution in ethanol is added dropwise under stirring with a magnetic stirrer at 200 rpm at 22+/−1 degree Celsius. The vial was covered after the addition and the solution was stirred for 30 minutes.

### Dissolution Behavior of Inventive (E1-E3) and Comparative Examples (C1-C4)

Crystallization Test:

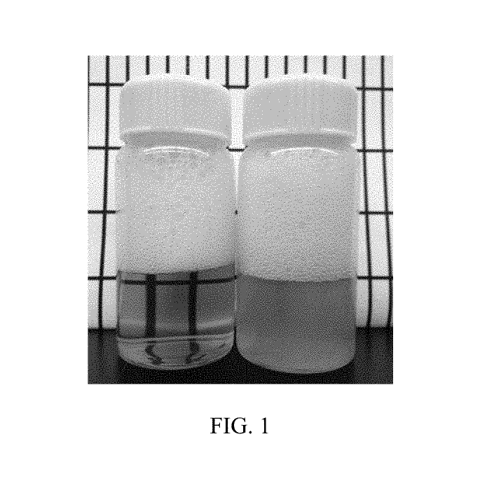

Presence of crystals in the test solutions was determined visually by observing a test grid through the sample solutions. Alteration of the test grid by the sample solution resulting in a blurred and hazy appearance indicated by the presence of crystals. FIG. 1 shows an example of a sample without crystals present (left) and an example of a sample with crystals present (right).

Comparative Examples C1-C4 and Inventive Examples E1-E3 are listed in Table 6, along with the results from the crystallization assessment (as measured in accord with the Crystallization Test as described above).

The results for C1 showed that allopregnanolone crystals remained and could not be dissolved, and, thus, were not incorporated into water. The results for C2 showed that allopregnanolone crystals remained and could not be dissolved, and, thus, were not incorporated, into an aqueous VaxSap saponin solution with 5 wt % VaxSap saponin surfactant. The results for C3 and C4 showed that when allopregnanolone solutions in ethanol were added to water under stirring, crystals of allopregnanolone were formed, and, thus, allopregnanolone could not be dissolved and were not incorporated into water using allopregnanolone solvent solutions. Crystals were formed for both 1600 and 400 ppm allopregnanolone.

The results for E1, E2, and E3 showed that when allopregnanolone solutions in ethanol were added to aqueous solutions of VaxSap saponin surfactant in accordance with the CSWC Method, crystals of allopregnanolone were absent. Thus, allopregnanolone was incorporated into aqueous VaxSap saponin surfactant solutions using the CSWC Method.

TABLE 6
Evaluation of crystal formation in comparative samples
1-4 (C1-C4) and inventive samples 1-3 (E1-E3).

| | C1 | C2 | C3 | C4 | E1 | E2 | E3 |
|---|---|---|---|---|---|---|---|
| Surfactant wt % | | | | | | | |
| VaxSap Saponins | | 5 | | | 4.2 | 4.2 | 1 |
| DI water | q.s. | q.s. | q.s. | q.s. | q.s. | q.s. | q.s. |
| AP Source | CS | CS | CS | CS | CS | CS | CS |
| (CarboSynth, | | | | | | | |
| Steraloids) | | | | | | | |
| AP added as | 1600 | 1600 | | | | | |
| crystals ppm | | | | | | | |
| AP in EtOH wt % | | | 1.6 | 1.6 | 1.6 | 1 | 1 |
| AP in final | | | 1600 | 400 | 1600 | 1250 | 400 |
| composition ppm | | | | | | | |
| EtOH wt % | | | 10 | 2.5 | 10 | 12.5 | 2.5 |
| visible crystals | Yes | Yes | Yes | Yes | No | No | No |

AP: Allopregnanolone;
DI: deionized;
ppm: parts per million;
EtOH: ethanol;
wt: weight;
CS: CarboSynth LLC

### Dissolution Behavior of Inventive Examples (E4-E7)

Inventive Examples E4-E7 are listed in Table 7, along with the results from the crystallization assessment (as measured in accord with the Crystallization Test as described above).

The results for E4, E5, E6, and E7 show that when allopregnanolone solutions in ethanol were added to aqueous solutions of different types of saponin surfactant in accordance with the CSWC Method, crystals of allopregnanolone were absent. Specifically, VaxSap saponin (E4), Soyasaponin I (E5), Ginsenoside Rg1 (E6), and Ginsenoside (E7) were used. Thus, allopregnanolone was incorporated into aqueous saponin surfactant solutions of various saponin surfactant types and origins.

TABLE 7
Evaluation of crystal formation in comparative samples 5-8 (C5-C8) and
inventive samples 4-7 (E4-E7)

| | E4 | E5 | E6 | E7 | C5 | C6 | C7 | C8 |
|---|---|---|---|---|---|---|---|---|
| Saponin surfactant wt % | | | | | | | | |
| VaxSap saponins | 1 | | | | | | | |
| Soyasaponin I | | 0.49 | | | | | | |
| Ginsenoside Rg1 | | | 1 | | | | | |
| Ginsenoside Rb1 | | | | 1 | | | | |
| H-b-cyclodextrin wt % | | | | | 3 | 1 | | |
| DI water | q.s. | q.s. | q.s. | q.s. | q.s. | q.s. | — | — |
| Propylene glycol | — | — | — | — | — | — | q.s. | — |
| Castor oil | — | — | — | — | — | — | — | q.s. |
| AP source (CarboSynth, | ST | ST | CS | CS | CS | CS | CS/ST | CS |
| Steraloids) | | | | | | | | |
| AP in EtOH wt % | 1 | 1 | 1 | 1 | 1 | 1 | 1 | 1 |
| AP in final composition ppm | 317 | 296 | 340 | 314 | 320 | 330 | 303 | 313 |
| EtOH wt % | 3.1 | 2.9 | 3.4 | 3.1 | 3.2 | 3.2 | 3 | 3 |
| visible crystals | No | No | No | No | No | Yes | No | No |

AP: Allopregnanolone;
DI: deionized;
ppm: parts per million;
EtOH: ethanol;
wt: weight;
CS: CarboSynth LLC (UK);
ST: Steraloids Inc. (USA)

### Dissolution Behavior of Comparative Examples (C5 and C6)

Comparative Examples C5 and C6 are listed in Table 7, along with the results from the crystallization assessment (as measured in accord with the Crystallization Test as described above).

The results for C5 confirm reports in the literature regarding the capability of h-b-cyclodextrin to render allopregnanolone water-soluble (Irwin, R W et al., “Allopregnanolone Preclinical Acute Pharmacokinetic and Pharmacodynamic Studies to Predict Tolerability and Efficacy for Alzheimer's Disease,” PLoS ONE 10(6): e0128313 (2015); “New Perspectives in Neurosteroids Action: a Special Player allopregnanolone” in Frontiers in Cellular Neuroscience, edited by Valerio Magnaghi, Giulia Puja, 2015). C5 contained 3 wt % h-b-cyclodextrin and 320 ppm allopregnanolone and crystals were not observed. The result for C6—presence of crystals—indicated that 1 wt % h-b-cycicodextrin was not sufficient to dissolve around 300 ppm allopregnanolone. Comparison to Inventive Examples E3-E7 indicated that saponin surfactants were more efficient in incorporating allopregnanolone into aqueous solution compared to h-b-cyclodextrin. The weight ratio of cyclodextrin to allopregnanolone in C5 was 94 to 1, in C6 it was 30 to 1. The presence of crystals in C6 showed that the ratio of cyclodextrin to allopregnanolone has to be higher than 30 to 1 to successfully incorporate allopregnanolone into aqueous solution using h-b-cyclodextrin. For E3-E7, the weight ratios of saponin surfactant to allopregnanolone were 25 to 1, 32 to 1, 34 to 1, 29 to 1, and 32 to 1 and for each of them crystals were absent.

### Dissolution Behavior of Comparative Examples (C7 and C8)

Comparative Examples C7 and C8 are listed in Table 2, along with the results from the crystallization assessment (as measured in accord with the Crystallization Test as described herein).

The results for C7 and C8 confirmed reports in the literature regarding the capability of propylene glycol and of castor oil to dissolve alloprenanolone. Crystals were absent for both C7 and C8.

Release of Allopregnanolone from Compositions Through a Membrane

It was discovered that allopregnanolone incorporated into saponin micelles was released significantly slower compared to allopregnanolone incorporated into a solvent in a monomolecular way and compared to h-b-cyclodextrin-allopregnanolone (CD-AP) complexes in water. (Monomolecular way refers to the ability of a solvent to dissolve a solid by separating the molecules of the solid and generating a clear and stable solution of individual molecules of the solid in a matrix of solvent molecules).

According to the literature (Irwin and Brinton, “Allopregnanolone as regenerative therapeutic for alzheimer's disease: Translational development and clinical promise,” Progress in Neurobiology 113:40-55 (2014); Irwin et al., “Frontiers in therapeutic development of allopregnanolone for Alzheimer's disease and other neurological disorders,” Frontiers in Cellular Neuroscience 8:203 (2014)), the CD-AP complexes consist of 2 molecules cyclodextrin and 1 molecule allopregnanolone per complex. Thus, such a complex has a molecular weight of around 2,700 g/mol and a hydrodynamic radius Rₕ similar to Rₕ of cyclodextrin and allopregnanolone.

Saponin micelles are larger compared to a allopregnanolone molecule and compared to a CD-AP complex and thus, have a larger Rₕ (Rₕ˜3 nm) and a larger molecular weight (>50,000 g/mol). It will be understood by those skilled in the art that allopregnanolone inside a saponin micelle has a significantly reduced diffusion coefficient compared to monomolecular allopregnanolone and to CD-AP complexes (due to the larger Rₕ) and, importantly, the release of allopregnanolone from the inside of a micelle into the aqueous phase is slow. Thus, the saponin micelles containing allopregnanolone can be seen as reservoirs for allopregnanolone allowing delivery of relatively large amounts of allopregnanolone combined with a prolonged release, but avoiding high peak concentrations of released allopregnanolone.

### Preparation of Comparative Examples C9-C10

Comparative Example C9 was prepared as follows: To 2 g solution of H-b-Cyclodextrin in DI water placed in a 20 mL scintillation vial the appropriate amount of an allopregnanolone solution in ethanol was added dropwise under stirring with a magnetic stirrer at 200 rpm at 22+/−1 degree Celsius. The vial was covered after the addition and the solution was stirred for 30 minutes.

Comparative Example C10 was prepared as follows: To 79.6 g DI water placed in a 4 ounce glass jar the appropriate amount of an allopregnanolone solution in ethanol was added dropwise under stirring with a magnetic stirrer at 200 rpm at 22+/−1 degree Celsius. The jar was covered after the addition and the solution was stirred for 30 minutes.

### Dialysis Behavior of Inventive (E4, E5) and Comparative Examples (C9 and C10)

Dialysis Test

Dialysis was done using Spectra/Por® 32 millimeter (mm) membrane tubing with a molecular weight cut-off of MWCO: 12,000-14,000, order#08-667D from Spectrum® Laboratories, Inc. (Rancho Dominguez, Calif.). 1 gram (g) donor solution was pipetted into a prewetted dialysis bag and placed into 100 g DI water bath (receiver solution). The water bath was stirred at 10 rpm using a magnetic stirrer and kept at 22+/−1 degree Celsius for 24 hours. Aliquots of 0.5 g of the dialysis receiver solution were taken after 1.66, 3.08, 8, 11.5, and after 24 hours for samples with saponin surfactant and cyclodextrin. The dialysis receiver solution samples were stored at −18 degree Celsius until further preparation for the allopregnanolone Quantification Test. After thawing, all dialysis receiver solution samples were prediluted with DI water and fully diluted with Arbor Assay® buffer X067-28 at a ratio of 1:3 (solution:buffer) to adjust the estimated allopregnanolone concentrations into the middle of the allopregnanolone Quantification Test standard curve concentrations. The solutions were used within 1 hour after thawing in the allopregnanolone Quantification Test.

To determine the kinetics of release of allopregnanolone through the dialysis membrane, the obtained relative concentrations of allopregnanolone in the bath over time were analyzed. With a constant diffusion coefficient and negligible concentration gradients within the donor and receiving solutions, first order kinetics were applied. Thus, the data were fitted using formula 1:

C_{rel.,bath}=1−C_{rel.,donor}·e^{-P·time},

with the relative allopregnanolone concentration C_{rel.,bath} in the bath (receiving solution), the relative allopregnanolone concentration C_{rel.,donor} in the donor solution at time=0, the permeation coefficient P as the adjustable parameter, and the time.

Allopregnanolone Quantification Test

Determination of allopregnanolone concentrations in dialysis and tissue permeation receiver solutions was done using DetectX® Allopregnanolone Enzyme Immunoassay Kit K044-H5 from Arbor Assays™ (Ann Arbor, Mich.). The DetectX® Allopregnanolone Immunoassay kit is designed to quantitatively measure allopregnanolone present in extracted serum, plasma, or dried fecal samples, in diluted urine and tissue culture media samples. Arbor Assays™ protocol described in K044-H1/H5, 160829, 2016 was followed.

A primary incubation of 2 hours at room temperature with shaking was applied. At the end of the incubation period the plate was washed and substrate was added. The substrate reacted with the bound allopregnanolone-peroxidase conjugate. After a short incubation, the reaction was stopped and the intensity of the generated color was detected in a microtiter plate reader capable of measuring 450 nanometer (nm) wavelength. The concentration of the allopregnanolone in the sample was calculated. Allopregnanolone material used in the composition, dialysis and tissue permeation experiments was used as the allopregnanolone standard. Standard solutions with 50, 25, 12.5, 6.26, 3.13, 1.56, 0.78, and 0.39 nanogram per milliliter (ng/mL) were prepared and used.

The assay protocol used was as follows: (1) The plate layout sheet on the back page was used to aid in proper sample and standard identification. The number of wells to be used was determined and unused wells were returned to the foil pouch with desiccant. The ziploc plate bag was sealed and stored at 4° C. (2) 50 microliter (μL) of samples or standards were pipetted into wells in the plate. (3) 75 μL of Assay Buffer was pipetted into the non-specific binding (NSB) wells. (4) 50 μL of Assay Buffer was pipetted into the maximum binding (Zero standard) wells. (5) 25 μL of the DetectX® Allopregnanolone-Conjugate was added to each well using a repeater pipet. (6) 25 μL of the DetectX® Allopregnanolone Antibody was added to each well, except the NSB wells, using a repeater pipet. (7) The sides of the plate were gently tapped to ensure adequate mixing of the reagents. The plate was covered with the plate sealer. (8) The plate was shaken at room temperature for 2 hours. (9) At the end of the incubation time, the plate was aspirated and each well was washed 4 times with 300 μL wash buffer. The plate was tapped dry on clean absorbent towels. (10) 100 μL of the TMB Substrate was added to each well, using a repeater pipet. (11) The plate was incubated at room temperature for 30 minutes without shaking. (12) 50 μL of the Stop Solution was added to each well, using a repeater pipet. (13) The optical density generated from each well was read in a plate reader capable of reading at 450 nm. (14) Allopregnanolone concentration was calculated for each sample using the standard curve generated from the standard solutions.

All samples were measured in duplicates. FIG. 2 shows the used standard curve. Solution concentrations were calculated with linear regression fitting. The size of the symbols in the figure was representative of the standard deviation.

Comparative Examples C9 and C10, as well as Inventive Examples E4 and E5 are listed in Table 8, along with the results for the permeation coefficient P (determined as defined in Dialysis Test).

TABLE 8

| | E4 | E5 | C9 | C10 |
|---|---|---|---|---|
| Donor Liquids: | | | | |
| Surfactant wt % | | | | |
| VaxSap Saponins | 1 | | | |
| Soyasaponin I | | 0.49 | | |
| H-b-Cyclodextrin wt % | | | 3.0 | |
| DI water | q.s. | q.s. | q.s. | q.s. |
| AP Source (Steraloids) | ST | ST | ST | ST |
| AP dissolved in EtOH at: | 1 wt % | 1 wt % | 1 wt % | 1 wt % |
| AP in final composition | 317 ppm | 296 ppm | 290 ppm | 4.8 ppm |
| EtOH | 3.1 wt % | 2.9 wt % | 2.9 wt % | 0.05 wt % |
| Dialysis: | | | | |
| Donor Liquid (g) | 1 | 1.03 | 1.01 | 1.01 |
| Bath mass (g), DI water | 100.87 | 100.69 | 100.62 | 100.59 |
| Permeation coefficient | 0.01 | 0.01 | 0.125 | 0.2 |
| P (1/h) | | | | |

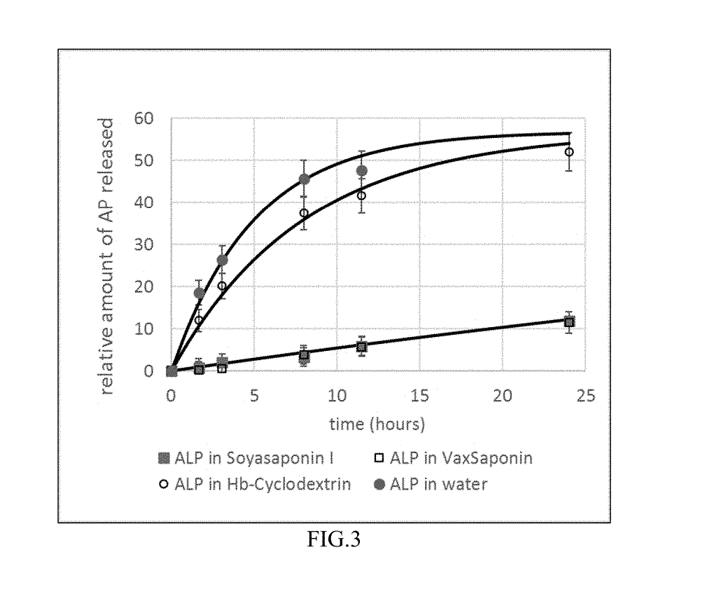

The permeation coefficient P obtained for the allopregnanolone in saponin-water solutions was significantly lower compared to the pure water and the cyclodextrin-water systems, i.e. the permeation of allopregnanolone through the membrane was significantly slower. The relative amounts of allopregnanolone released over time for systems with or without saponin are provided in FIG. 3.

It will be appreciated by those skilled in the art that changes could be made to the embodiments described above without departing from the broad inventive concept thereof. It is understood, therefore, that this invention is not limited to the particular embodiments disclosed, but it is intended to cover modifications within the spirit and scope of the present invention as defined by the present description.

It is claimed:

1. A composition comprising:
a. a neurosteroid; and
b. a saponin in an amount effective to form a self-assembled structure incorporating the neurosteroid.

2. The composition of claim 1, wherein the self-assembled structure is selected from the group consisting of a micelle, a gel, a liposome, a lamellar phase, and a multi-lamellar vesicle.

3. The composition of claim 2, wherein the self-assembled structure is a micelle.

4. The composition of claim 3, wherein the saponin is selected from the group consisting of a soyasaponin, a quillaja saponin, and a ginsenoside saponin.

5. The composition of claim 4, wherein the neurosteroid is selected from the group consisting of an allopregnanolone, a tetrahydrodeoxycorticosterone (THDOC), and a progesterone.

6. The composition of claim 5, wherein the neurosteroid is an allopregnanolone.

7. The composition of claim 6, wherein the saponin is at least about 0.1 wt % relative to the total weight of the composition.

8. The composition of claim 7, wherein the composition comprises at least about 100 parts per million of the neurosteroid.

9. The composition of claim 8, wherein the composition further comprises a pharmaceutically acceptable carrier.

10. The composition claim 9, wherein the composition further comprises an adjuvant.

11. The composition of claim 10, wherein the composition has a permeation coefficient P of about 0.01 per hour to about 0.05 per hour.

12. The composition of claim 11, wherein the composition has a permeation coefficient P of about 0.01 per hour.

13. A method of treating a perinatal depression (PND) in a subject in need thereof, the method comprising administering to the subject a therapeutically effective amount of the composition of claim 12.

14. The method of claim 13, wherein the therapeutically effective amount of the composition is administered topically, orally, mucosally, intravenously or by inhalation.

15. The method of claim 14, wherein the therapeutically effective amount of the composition is administered in a single dose.

16. The method of claim 15, wherein the therapeutically effective amount of the composition has a permeation coefficient P of about 0.01 per hour to about 0.05 per hour.

17. The method of claim 16, wherein the therapeutically effective amount of the composition has a permeation coefficient P of about 0.01 per hour.

18. The method of claim 17, wherein the therapeutically effective amount of the composition has a permeation coefficient P at least two times lower than a permeation coefficient P of a composition comprising a monomolecular neurosteroid or a cyclodextrin neurosteroid complex.

19. A method of producing the composition of claim 12, the method comprising adding a solution of the neurosteroid dissolved in an organic solvent to a saponin solution under flow, wherein the neurosteroid is incorporated into the self-assembled structure.

20. The method of claim 19, wherein the organic solvent dissolves at least 0.1 wt % of the neurosteroid.

21. The method of claim 20, wherein at least 1 wt % of the organic solvent is soluble in water.

22. The method of claim 21, wherein the organic solvent is selected from the group consisting of ethanol, methanol, propanol, butanol, glycol, ethylene glycol, propylene glycol, butylene glycol, diethyl ether, and mixtures thereof.

23. The method of claim 22, wherein the organic solvent is ethanol.
